SEQUENCE LISTING (2 sequences)

SEQ ID NO: 1 - protein, 27 aa, synthetic construct
  REGION 1..27: note = AH-D peptide
  SITE 1..27: note = D-amino acid
  SITE 27: note = C-terminal amidation
SGSWLRDVWDWICTVLTDFKTWLQSKL

SEQ ID NO: 2 - protein, 27 aa, synthetic construct
  REGION 1..27: note = NH-D peptide
  SITE 1..27: note = D-amino acid
  SITE 27: note = C-terminal amidation
SGSWLRDDWDWECTVLTDDKTWLQSKL

(19) United States
(12) Patent Application Publication
(10) Pub. No.: US 2023/0113262 A1
(43) Pub. Date: Apr. 13, 2023

(54) Peptide for cancer immunotherapy rupturing tumor-derived vesicle and use thereof

(71) Applicant: RESEARCH & BUSINESS FOUNDATION SUNGKYUNKWAN UNIVERSITY, Suwon-si (KR)

(72) Inventors: Jae Hyung PARK, Suwon-si (KR); Joshua Alexander JACKMAN, Suwon-si (KR); Hyewon KO, Daejeon (KR); Sol SHIN, Suwon-si (KR); Bo Kyeong YOON, Gwangju (KR); Chanho KIM, Suwon-si (KR); Soyoung SON, Gunpo-si (KR)

(73) Assignee: RESEARCH & BUSINESS FOUNDATION SUNGKYUNKWAN UNIVERSITY, Suwon-si (KR)

(21) Appl. No.: 17/818,874

(22) Filed: Aug. 10, 2022

(30) Foreign Application Priority Data
Aug. 10, 2021 (KR) 10-2021-0105661
Aug. 2, 2022 (KR) 10-2022-0096389

(51) Int. Cl. A61K 39/00; C07K 14/705

(52) U.S. Cl. CPC A61K 39/001102; C07K 14/705

(57) ABSTRACT

The present invention relates to a peptide for cancer immunotherapy that disrupts tumor-derived vesicles and a use thereof. The peptide according to the present invention has an α-helical structure, inhibits T-cell functional impairment caused by tumor-derived vesicles and controls the formation of a tumor microenvironment as an immunosuppressive environment by disrupting the tumor-derived vesicles, and has effects of not only enhancing cancer immunotherapy activity but also inhibiting the metastasis of cancer cells through co-administration with an immune checkpoint inhibitor. Additionally, when the peptide is modified with PEG through a pH-sensitive linker, it has effects of increasing the stability of the peptide in vivo, and disrupting tumor-derived vesicles under the pH condition of the tumor microenvironment. Accordingly, the peptide of the present invention is expected to be effectively used in a composition for cancer immunotherapy, a composition for enhancing the sensitivity of cancer immunotherapy, or the composition for inhibiting cancer metastasis.

### TECHNICAL FIELD

The present invention relates to a peptide for cancer immunotherapy, which disrupts tumor-derived vesicles, and a use thereof.

### Background Art of Invention

Cancer is one of the intractable diseases that mankind must solve, and a huge amount of capital is being invested in the development of cancer treatment worldwide. In Korea, cancer is the No. 1 cause of death of disease, and approximately 100,000 people or more are diagnosed with cancer and approximately 60,000 or more die from it yearly.

Currently, methods for treating cancer may be broadly divided into surgery, radiation therapy, chemotherapy, and biological therapy. Patients who cannot easily undergo surgery or radiation therapy among these methods (approximately 50% of all cancer cases), and patients who have metastasized cancer are generally treated with chemotherapy. However, due to drug resistance and the recurrence, metastasis, and sequelae of cancer, it is important to recently develop cancer treatment technology that can minimize side effects.

Recently, starting with the cancer immunotherapeutic Keytruda, unlike existing anticancer agents that attack cancer itself, an immunochemotherapy, which stimulates the body's immune system to selectively attack cancer cells, is attracting attention. Particularly, since immune checkpoint inhibitors, which treat cancer by blocking an immune evasion signal of cancer cells, show positive clinical results in various carcinomas, including malignant melanoma, there is ongoing research on these inhibitors as third-generation anticancer agents worldwide.

Cancer immunotherapy using an immune checkpoint inhibitor is a method of attacking cancer cells by activating T cells by blocking an immune evasion signal of an immune checkpoint molecule such as PD-1, PD-L1, or CTLA-4, expressed through an immune evasion mechanism for the survival of cancer cells using a corresponding antibody (PD-1 antibody or PD-L1 antibody). However, there is a limitation in that commercially available antibodies exhibit an anticancer effect in only 15 to 30% of patients, but not effective in the remaining patients. It is observed that these patients show significantly reduced T cell sensitization, activation, and T-cell tumor-penetrating ability.

In this process, various mechanisms involving tumor-derived vesicles secreted from tumor cells have been recently reported. According to reports, tumor-derived vesicles contribute to tumor angiogenesis and interstitial tissue formation, thereby adjusting the tumor microenvironment to an immunosuppressive environment, and administered antibodies are neutralized by the PD-L1 protein on the surface of tumor-derived vesicles circulating in the blood, thereby inhibiting the systemic T-cell action. Therefore, to increase the response rate to an immune checkpoint inhibitor, a strategy that can remove and inhibit tumor-derived vesicles is needed.

Meanwhile, a tumor consists of not only cancer cells but also various surrounding cells, such as stromal cells, fibroblasts, immune- and inflammation-mediating cells, vascular endothelial cells, or connective tissue-forming cells, which are exposed to hypoxic and acidic conditions. Such a special tumor microenvironment controls cancer cell proliferation, invasion and metastasis, thereby affecting not only the cancerous process but also drug resistance to anticancer treatment.

Therefore, the inventors intend to use a peptide that has an α-helix structure capable of controlling the tumor microenvironment and inhibiting T cell functional impairment by disrupting tumor-derived vesicles as a cancer immunotherapeutic agent or a combination therapy with an immune checkpoint inhibitor that can enhance the reaction sensitivity of cancer immunotherapy.

### DISCLOSURE OF INVENTION

### Technical Problem

The inventors have developed a peptide that can inhibit T-cell functional impairment caused by tumor-derived vesicles and control a tumor microenvironment by disrupting tumor-derived vesicles, and enhance the activity of cancer immunotherapy through co-administration with an immune checkpoint inhibitor, and based on this, the present invention was completed.

Therefore, the present invention is directed to providing a peptide for cancer immunotherapy, which comprises an amino acid sequence of SEQ ID NO: 1.

The present invention is also directed to providing a pharmaceutical composition for cancer immunotherapy, preventing or treating cancer, enhancing cancer immunotherapy sensitivity, or inhibiting cancer metastasis, which comprises the peptide as an active ingredient.

However, technical problems to be solved in the present invention are not limited to the above-described problems, and other problems which are not described herein will be fully understood by those of ordinary skill in the art to which the present invention belongs from the following descriptions.

### Technical Solution

To achieve the above-described purposes, the present invention provides a peptide for cancer immunotherapy, which comprises an amino acid sequence of SEQ ID NO: 1.

The present invention also provides a pharmaceutical composition for cancer immunotherapy, which comprises the peptide as an active ingredient.

The present invention also provides a pharmaceutical composition for preventing or treating cancer, which comprises the peptide as an active ingredient.

The present invention also provides a pharmaceutical composition for enhancing cancer immunotherapy sensitivity, which comprises the peptide as an active ingredient.

The present invention also provides a pharmaceutical composition for inhibiting cancer metastasis, which comprises the peptide as an active ingredient.

According to one embodiment of the present invention, the peptide may have an α-helical structure, but the present invention is not limited thereto.

According to another embodiment of the present invention, the peptide may disrupt tumor-derived vesicles, but the present invention is not limited thereto.

According to still another embodiment of the present invention, the peptide may inhibit T cell functional impairment, but the present invention is not limited thereto.

According to yet another embodiment of the present invention, the peptide may control a tumor microenvironment, but the present invention is not limited thereto.

According to yet another embodiment of the present invention, the peptide may inhibit tumor angiogenesis or fibrosis, but the present invention is not limited thereto.

According to yet another embodiment of the present invention, the peptide may comprise a peptide modified with polyethylene glycol (PEG), but the present invention is not limited thereto.

According to yet another embodiment of the present invention, the PEG may bind to the peptide via a linker, but the present invention is not limited thereto.

According to yet another embodiment of the present invention, the linker may be sensitive to a tumor microenvironment, but the present invention is not limited thereto.

According to yet another embodiment of the present invention, the linker may be a cleavable linker cleaved in response to a tumor microenvironment, but the present invention is not limited thereto.

According to yet another embodiment of the present invention, the pharmaceutical composition for cancer immunotherapy may further comprise an immune checkpoint inhibitor, but the present invention is not limited thereto.

According to yet another embodiment of the present invention, the immune checkpoint inhibitor may be one or more selected from the group consisting of a PD-1 inhibitor, a PD-L1 inhibitor, and a CTLA-4 inhibitor, but the present invention is not limited thereto.

According to yet another embodiment of the present invention, the cancer may be one or more selected from the group consisting of colorectal cancer, breast cancer, prostate cancer, melanoma, lung cancer, head and neck cancer, ovarian cancer, bladder cancer, stomach cancer, esophageal cancer, bile duct cancer, pancreatic cancer, liver cancer, cervical cancer, skin cancer, lymphoma, thyroid cancer, bone marrow cancer, endometrial cancer, and brain tumors, but the present invention is not limited thereto.

According to yet another embodiment of the present invention, the peptide may improve the cancer immunotherapy activity of the immune checkpoint inhibitor, but the present invention is not limited thereto.

According to yet another embodiment of the present invention, the peptide may inhibit pre-metastatic niche formation, but the present invention is not limited thereto.

In addition, the present invention provides a method of cancer immunotherapy or enhancing the sensitivity of cancer immunotherapy, which comprises administering the composition comprising a peptide represented by the amino acid sequence of SEQ ID NO: 1 as an active ingredient to a subject in need thereof.

In addition, the present invention provides a use of the composition comprising a peptide represented by the amino acid sequence of SEQ ID NO: 1 as an active ingredient for cancer immunotherapy or enhancing the sensitivity of cancer immunotherapy.

In addition, the present invention provides a use of a peptide represented by the amino acid sequence of SEQ ID NO: 1 for preparing a drug for cancer immunotherapy or enhancing the sensitivity of cancer immunotherapy.

In addition, the present invention provides a method of preventing or treating cancer, or inhibiting the metastasis of cancer, which comprises administering a composition comprising a peptide represented by the amino acid sequence of SEQ ID NO: 1 as an active ingredient into a subject in need thereof.

In addition, the present invention provides a use of a composition comprising a peptide represented by the amino acid sequence of SEQ ID NO: 1 as an active ingredient for preventing or treating cancer, or inhibiting the metastasis of cancer.

In addition, the present invention provides a use of a peptide represented by the amino acid sequence of SEQ ID NO: 1 for preparing a drug for preventing or treating cancer, or inhibiting the metastasis of cancer.

### Effects of Invention

A peptide according to the present invention has an α-helix structure, and inhibits T-cell functional impairment caused by tumor-derived vesicles and controls the formation of a tumor microenvironment as an immunosuppressive environment by disrupting the tumor-derived vesicles, and has effects of not only enhancing cancer immunotherapy activity but also inhibiting the metastasis of cancer cells through co-administration with an immune checkpoint inhibitor. In addition, when the peptide is modified with PEG through a pH-sensitive linker, it has effects of increasing the stability of the peptide in vivo, and disrupting tumor-derived vesicles under the pH condition of the tumor microenvironment. Accordingly, the peptide of the present invention is expected to be effectively used in a composition for cancer immunotherapy, a composition for enhancing the sensitivity of cancer immunotherapy, or the composition for inhibiting cancer metastasis.

### DESCRIPTION OF DRAWINGS

FIGS. 1A to 1E show biophysical evaluation results for AH-D and NH-D peptides according to one embodiment of the present invention, wherein FIG. 1A shows a circular dichroism spectroscopy result, FIG. 1B is the results confirming the cytotoxicity of the AH-D and NH-D peptides, and FIGS. 1C to 1E are the results confirming the real-time interaction of liposomes with the AH-D or NH-D peptides by QCM-D measurement, showing a corresponding resonance frequency (FIG. 1C), energy dissipation shift (FIG. 1D), and a time-dependent plot of energy dissipation shift as the function of a resonance frequency shift (FIG. 1E), respectively.

FIGS. 2A and 2B show the results of confirming the characteristics of a tumor-derived exosome (T-EXO) according to one embodiment of the present invention, and show the size distribution of T-EXO (FIG. 2A) and Western blotting of major protein markers for the exosome (FIG. 2B), respectively.

FIGS. 2C to 2I show the results confirming a tumor-derived exosome (T-EXO) disruption effect caused by AH-D peptide treatment through nanoparticle tracking analysis (NTA) according to one embodiment of the present invention, and show the change in T-EXO concentration by AH-D peptide treatment for different cell types (FIG. 2C), the result that confirms PD-L1^{EXO} expression in B16F10 cells (FIG. 2D), the change in T-EXO concentration by AH-D peptide treatment under various time conditions (FIG. 2E), the change in T-EXO concentration by AH-D peptide treatment under various pH concentrations (FIG. 2F), the change in T-EXO concentration according to an AH-D peptide treatment concentration (FIG. 2G), a set of NTA images (FIG. 2H), and a set of transmission electron microscope images (FIG. 2I), respectively.

FIGS. 3A to 3H confirm the liposome rupture activity of the AH-D peptide according to one embodiment of the present invention, which show the QCM-D resonance frequency (FIG. 3A) and energy dissipation shift (FIG. 3B), normalized as a function of time for the AH-D peptide-induced rupture of surface-adhered liposomes under various pH conditions; a time-dependent plot of energy dissipation as the function of a resonance frequency shift (FIG. 3C), the AH-D peptide-induced liposome rupture time according to a peptide concentration under various pH conditions (FIG. 3D), the QCM-D resonance frequency (FIG. 3E) and energy dissipation signals (FIG. 3F) by AH-D peptide treatment for a surface-adhered liposome layer under various pH conditions, AH-D peptide binding cooperativity according to various pH conditions (FIG. 3G), and the Gibbs free energy of membrane partitioning for AH-D peptide under various pH conditions (FIG. 3H).

FIG. 4A is a schematic diagram of the action of a PEG-linker-modified peptide according to one embodiment of the present invention, and FIGS. 4B to 4D are the results of analyzing the characteristics of a PEG-linker and a PEG-linker-modified peptide according to one embodiment of the present invention, which show the synthesis of PEG-linkers (FIG. 4B), the result of measuring circular dichroic spectroscopy for a PEG-modified peptide and a PEG-linker-modified peptide (FIG. 4C), and the result of measuring dissociation over time for PEG and peptides according to pH (FIG. 4D), respectively.

FIGS. 5A to 5I show a T-EXO-mediated CD8⁺ T cell functional impairment inhibitory effect of AH-D peptide according to one embodiment of the present invention, wherein FIG. 5A is the diagram of PD-1 binding to T-EXO and its action thereof, and FIGS. 5B to 5H show a PD-L1 content in T-EXO isolated from the plasma of a B16F10 tumor-bearing mouse (FIG. 5B) and the PD-1 binding ability of T-EXO (FIG. 5C), a set of confocal microscope images for evaluating endocytic uptake in vivo of AH-D peptide-treated T-EXO (FIG. 5D), and the result of quantifying the relative fluorescence intensity over time (FIG. 5E), the result of measuring CD8⁺ T cell proliferation (FIG. 5F) and the result of confirming the recovery of a CD8⁺ T cell function (FIG. 5G) according to AH-D peptide treatment through flow cytometry, the result of confirming the increase in pre-inflammatory cytokine production according to AH-D peptide treatment (FIG. 5H), and the ratio of Ki-67-expressing T cells as a cell proliferation marker (FIG. 5I).

FIGS. 6A to 6G show the T-EXO-mediated T cell functional impairment inhibitory effect of a PEG-linker-modified peptide according to one embodiment of the present invention, and show the schematic diagram of the process of a PD-1/PD-L1 binding ability analysis experiment to confirm a T-EXO disruption effect (FIG. 6A), the result that confirms the change in T-EXO concentration according to PEG-linker-modified peptide treatment under various pH concentration conditions (FIG. 6B), the result that measures a PD-L1 level on an exosome surface according to PEG-linker-modified peptide treatment (FIG. 6C), the result of confocal microscope imaging to evaluate the cellular uptake behavior of T-EXO for T cells (FIG. 6D), the result of quantifying the experimental result of FIG. 6D (FIG. 6E), and the result that confirms CD8⁺ T cell proliferation behavior according to PEG-linker-modified peptide treatment (FIGS. 6F and 6G), respectively.

FIGS. 7A and 7B show the cytotoxicity of an AH-D peptide, a PEG-modified peptide, and a PEG-linker-modified peptide in a B16F10 cell line (FIG. 7A) and a NIH3T3 cell line (FIG. 7B) according to one embodiment of the present invention.

FIG. 8 shows a blood T-EXO level when a tumor animal model was treated with a PEG-linker-modified peptide according to one embodiment of the present invention.

FIGS. 9A to 91 show the anti-tumor effect synergistic action by blocking of the interaction of AH-D peptide with PD-L1 and PD-1 according to one embodiment of the present invention, and show a PD-L1^{EXO} level circulated in vivo in AH-D peptide treatment (FIG. 9A), CD4⁺CD25⁺ regulatory T cells (Tregs), CD11b⁺Gr-1⁺ bone marrow-derived suppressor cells (MDSCs), and TGF-β levels in AH-D peptide treatment (FIG. 9B), an experimental protocol for evaluating the anti-tumor efficacy of combination therapy of the AH-D peptide and aPD-1 antibody (FIG. 9C), a tumor volume (FIG. 9D) and a weight (FIG. 9E) in co-administration of the AH-D peptide and the aPD-1 antibody, H&E staining (upper) and MT staining (lower) results according to the co-administration of the AH-D peptide and the aPD-1 antibody in cancer tissue (FIG. 9F), H&E staining results for major organs (FIG. 9G), the change in body weight of a mouse (FIG. 9H), and immunohistochemical results for α-smooth muscle actin (α-SMA), fibronectin, and the CD31 marker of tumor tissue (FIG. 9I), respectively.

FIGS. 10A to 10E show the anti-tumor effect synergistic action according to co-administration of the AH-D peptide and aPD-1 antibody according to one embodiment of the present invention, and show a PD-L1^{EXO} level according to the co-administration of the AH-D peptide and the aPD-1 antibody (FIG. 10A), the number of CD8⁺ cells penetrated into tumor tissue (FIG. 10B), the distribution of CD3⁺ CD8⁺ T cells confirmed by flow cytometry (FIG. 10C), the degree of T cell proliferation (FIG. 10D), and the frequency of (GrzB)-expressing CD8⁺ T cells (FIG. 10E), respectively.

FIGS. 11A to 11E show the cancer pre-metastatic niche formation inhibitory effect of T-EXO by the AH-D peptide according to one embodiment of the present invention, and show the result of migration analyses according to T-EXO and AH-D peptide treatment in vitro (FIG. 11A) and the result of measuring the activation of CAF cells involved in cancer metastasis according to T-EXO and AH-D peptide treatment in vitro (FIG. 11B) in order to evaluate the metastatic probability of cancer cells, an experimental protocol for confirming the anti-metastatic effect of the AH-D peptide in vivo (FIG. 11C), the result of confirming whether or not to metastasize after the lungs are extracted (FIG. 11D), and the histological analysis result for a lung tissue-metastatic lesion and the immunohistochemical staining result for a lung tissue section (FIG. 11E), respectively.

FIG. 12 illustrates the strategy of lipid envelope exosome disruption (LEED) according to one embodiment of the present invention.

### DETAILED DESCRIPTION OF INVENTION

In one experimental example of the present invention, it was confirmed that the AH-D peptide according to the present invention disrupts tumor-derived vesicles (T-EXO), and exhibited membrane disruption enhanced under an acidic condition related to a tumor microenvironment (see Experimental Example 1).

In another experimental example of the present invention, as a result of analyzing the characteristics of PEG- and PEG-linker-modified peptides according to the present invention, it was confirmed that the modified peptides also have a structure capable of disrupting the phospholipid membrane of an exosome, and it can be seen that PEG-linker-modified peptide responds to a tumor microenvironment by confirming linker disruption in response to a weakly acidic environment (see Experimental Example 2).

In still another experimental example of the present invention, the AH-D peptide according to the present invention inhibits T cell functional impairment, for example, inhibits PD-L1^{EXO} binding ability to T cells, increases the proliferation probability of T cells, inhibits the suppression of the cytotoxic function of T cells by T-EXO and increases the expression of a pre-inflammatory cytokine (see Experimental Example 3).

In yet another experimental example of the present invention, it was confirmed that a linker of PEG-linker-modified peptide according to the present invention is disrupted only at pH 6.5, which is a tumor microenvironment, and the AH-D peptide is released, thereby exhibiting the T-EXO disruption effect, and confirmed that, at pH 6.5 due to the T-EXO membrane disruption, PD-1/PD-L1 binding is inhibited, PD-1-mediated cellular absorption to the surface of a T cell membrane is inhibited, and a T cell functional impairment reducing effect by T-EXO is exhibited (see Experimental Example 4).

In yet another experimental example of the present invention, it was confirmed that all of the AH-D peptide, PEG-modified peptide, and PEG-linker-modified peptide according to the present invention do not exhibit cytotoxicity in B16F10 and NIH3T3 cell lines (see Experimental Example 5).

In yet another experimental example of the present invention, as a result of intravenous injection of PEG-linker-modified peptide according to the present invention into a tumor animal model, it was confirmed that a tumor tissue T-EXO ratio was decreased by approximately 45% (see Experimental Example 6).

In yet another experimental example of the present invention, it was confirmed that, in the single treatment of the AH-D peptide according to the present invention, the levels of blood PD-L1^{EXO} secreted from a tumor and circulated and immunosuppressive cells in tumor tissue are reduced, and more excellent anti-tumor efficacy is exhibited in combined treatment of the AH-D peptide and an anti PD-1 antibody (aPD-1). In addition, it was confirmed that the AH-D peptide more immunoactively reconstructs the tumor microenvironment and inhibits tumor fibrosis and angiogenesis (see Experimental Example 7).

In yet another experimental example of the present invention, it was confirmed that the AH-D peptide of the present invention inhibits pre-metastatic niche formation, and effectively inhibits the metastasis promoting function of T-EXO (see Experimental Example 8).

Therefore, the present invention provides a peptide for cancer immunotherapy, which comprises an amino acid sequence of SEQ ID NO: 1.

In the present invention, the “peptide” refers to a polymer consisting of two or more amino acids by a peptide bond, and the peptide may have an α-helical structure. In the present invention, when the peptide has a random coil secondary structure, it may not exhibit activity that the peptide of the present invention exhibits, such as the activity of disrupting tumor-derived vesicles, and in one example or experimental example of the present invention, the peptide having a random coil secondary structure was used as a control. In addition, the peptide comprises the sequence of 27 amino acids such as SGSWLRDVWDWICTVLTDFKTWLQSKL (SEQ ID NO: 1), and an amine group (—NH₂) may be comprised at the C-terminus of the amino acid sequence. The peptide of the present invention may be prepared by a chemical synthesis method known in the art, along with a molecular biology method.

The peptide of the present invention may be a variant or fragment having a different sequence by the deletion, insertion, substitution of an amino acid residue or a combination thereof in a range that does not affect the activity of the peptide. Considering variations with biologically equivalent activity as above, the peptide of the present invention is interpreted to comprise an amino acid sequence having substantial identity to the amino acid sequence of SEQ ID NO: 1. The substantial identity may refer to an amino acid sequence having 75% or more, preferably, 80% or more, more preferably 90% or more, and most preferably 95% or more sequence homology when the amino acid sequence of the present invention is aligned to correspond as close as possible to any other amino acid sequence, and the aligned amino acid sequences are analyzed using an algorithm commonly used in the art. In addition, the peptide may further comprise a targeting sequence, a tag, a labeled residue, or an amino acid sequence prepared for a specific purpose to increase a half-life or peptide stability, and in order to acquire better chemical stability, enhanced pharmacological characteristics (half-life, absorption, potency, efficacy, etc.), modified specificity (e.g., targetability to a specific site), or reduced antigenicity, a protective group such as polyethylene glycol (PEG) may be additionally bound to the N-terminus or C-terminus of the peptide, but the present invention is not limited thereto.

In the present invention, the peptide may comprise a polyethylene glycol (PEG)-modified peptide, and here, the PEG may be bound to a peptide via a linker (in the form of a PEG-linker), but the present invention is not limited thereto.

In the present invention, the peptide may additionally have an amine group (—NH₂) at the C-terminus, the PEG-linker may be bound by the reaction with an amine group added to the C-terminus of the peptide or an amine group of the N-terminus thereof, and according to one example or experimental example of the present invention, at both of the C-terminus and the N-terminus of the peptide, an amine group may be bound, but the present invention is not limited thereto.

The “modification” used herein refers to the formation of a modification layer by binding PEG to the peptide according to the present invention or coating the peptide according to the present invention with PEG, and the binding may be a chemical bond, such as an ionic bond, a covalent bond, a metallic bond, a coordinate bond, a hydrogen bond, and an intermolecular force, but the present invention is not limited thereto.

The “linker” used herein refers to a compound for connecting the PEG to the peptide according to the present invention.

In the present invention, the linker may be responsive to a tumor microenvironment, and more specifically, may be a cleavable linker that is cleaved in response to a tumor microenvironment, but the present invention is not limited thereto.

The “tumor microenvironment” used herein refers to an environment in which a tumor is present and which is a non-cellular region and a region directly extending from tumorous tissue but does not belong to the intracellular compartment of a cancer cell itself, and is the comprehensive concept that collectively refers to not only the group of constituent cells, such as vascular cells, stromal cells, and immune cells, present in the tumor, but also its environment (weak acidification and hypoxia). Tumors and tumor microenvironments are closely related and constantly interact. Tumors may change their microenvironments, and their microenvironments may affect how a tumor grows and spreads. A tumor microenvironment may show a weakly acidic pH, have lower concentrations of glucose and other nutrients compared to plasma, and have a high concentration of lactic acid and a temperature that is 0.3° C. to 1° C. higher than hypoxic and normal physiological temperatures.

In the present invention, the linker may further comprise all linkers designed to cleave in response to conditions, such as pH, ROS, an enzyme, hypoxia, and a temperature, which are characteristic of the tumor microenvironment distinct from normal tissue, but the present invention is not limited thereto.

A peptide may be released by the cleavage of the linker in the tumor microenvironment to exhibit the disruptive effect of tumor-derived exosomes.

According to one example or experimental example of the present invention, the cleavable linker may be pH responsive, that is, responsive to hydrolysis at a specific pH value. Typically, the pH-responsive linker is hydrolysable under an acidic condition. For example, an acid-labile linker, which is hydrolysable in lysosomes, for example, 3-(4-methyl-2,5-dioxo-2,5-dihydrofuran-3-yl)propanoic acid, hydrazone, semicarbazone, thiosemicarbazone, cis-aconitic amide, orthoester, acetal, or ketal. Another example of the cleavable linker may be a dimethyl maleic anhydride derivative, such as 2-propionic-3-methylmaleic anhydride (carboxylated dimethyl maleic anhydride or CDM). According to one example or experimental example of the present invention, the linker may comprise 3-(4-methyl-2,5-dioxo-2,5-dihydrofuran-3-yl)propanoic acid, and the linker may form a PEG-CDM-peptide by producing a carboxy-dimethylmaleic amide (CDM) bond by binding to an amine group of a peptide, but the present invention is not limited thereto.

Since such a linker is relatively stable under neutral pH conditions, for example, under pH conditions in blood, it may be unstable at an acidic pH of the tumor microenvironment, so it can be cleaved.

In the present invention, the pH of the tumor microenvironment may be, for example, pH 5 to 6.9, pH 5 to 6.8, pH 5 to 6.5, pH 5.3 to 6.9, pH 5.3 to 6.8, pH 5.3 to 6.5, pH 5.5 to 6.9, pH 5.5 to 6.8, pH 5.5 to 6.5, pH 5.8 to 6.9, pH 5.8 to 6.8, pH 5.8 to 6.5, pH 6 to 6.9, pH 6 to 6.8, pH 6 to 6.5, pH 6.3 to 6.9, pH 6.3 to 6.8, pH 6.3 to 6.5, pH 6.5 to 6.9, pH 6.5 to 6.8, pH 6.5, or pH 6.8, but the present invention is not limited thereto.

In the present invention, the linker may be a cleavable linker that is cleaved by a protease. The protease may be an intracellular peptidase or protease, as well as a lysosome or endosome protease, and may be, for example, cathepsin B, cathepsin K, matrix metalloproteinase (MMP), urokinase, or plasmin, but the present invention is not limited thereto.

Such a linker may be a peptide linker. A peptide, which is a constituent of the peptide linker, may include 20 major amino acids well known in the biochemistry field and minor amino acids, for example, two or more amino acid residues comprising citrulline. The amino acid residues comprise all stereoisomers, and may be a D- or L-conformation.

For example, the peptide may be an amino acid unit comprising 2 to 12 amino acid residues independently selected from glycine, alanine, phenylalanine, lysine, arginine, valine and citrulline. As an exemplary peptide linker, a Val-Cit linker or a Phe-Lys dipeptide may be included.

In the present invention, the linker may include a spacer domain for binding a linker to an antibody. For example, the linker may include a reactive site with an electrophilic group reactive to a nucleophilic group on an antibody as a spacer domain. The electrophilic group on the linker provides a convenient linker-attachment site for an antibody.

A useful nucleophilic group on an antibody comprises, for example, sulfhydryl, hydroxyl and amino groups. A heteroatom of the nucleophilic group of an antibody is reactive to the electrophilic group on the linker, and forms a covalent bond with the linker. A useful electrophilic group of the linker may be, for example, a maleimide (e.g., malimidocaproyl) group and a haloacetamide group.

In addition, the linker may include a reactive site with a nucleophilic group reactive to an electrophilic group present on an antibody as a spacer domain. The electrophilic group on an antibody provides a convenient attachment site for the linker. A useful electrophilic group on an antibody comprises, for example, a carbonyl group of an aldehyde or a ketone, or a carboxyl group.

The heteroatom of the nucleophilic group of the linker may react with an electrophilic group on an antibody, and may form a covalent bond to an antibody. A useful nucleophilic group of the linker may be, for example, a hydrazide group, an oxime group, an amino group, a hydrazine group, a thiosemicarbazone group, a hydrazine carboxylate group, or an aryl hydrazide group. An electrophilic group on the antibody provides a convenient attachment site for the linker.

In addition, the linker may include a self-immolative site (e.g., p-aminobenzyl alcohol (PABA), p-aminobenzyl oxycarbonyl (PABC), or PAB-OH).

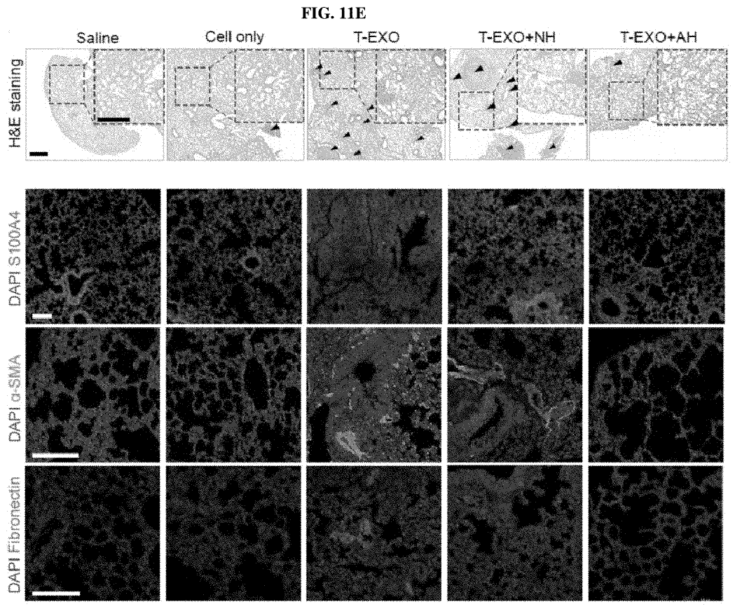

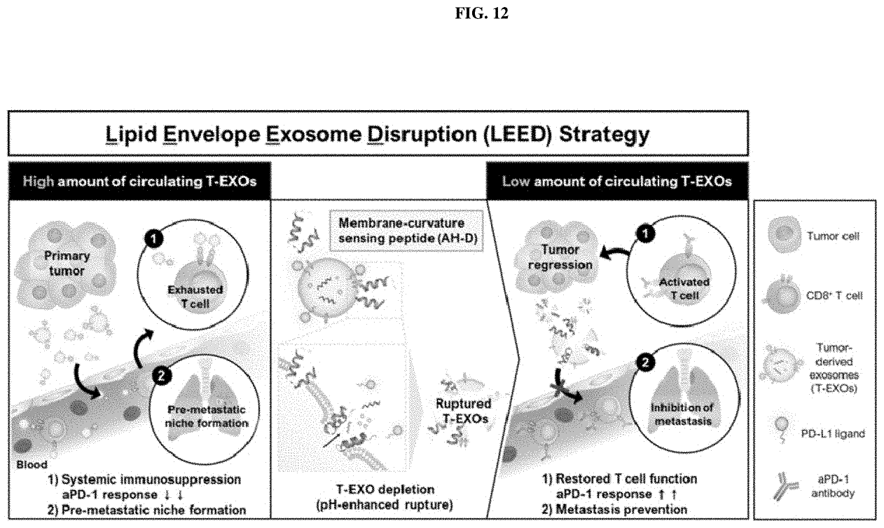

In the present invention, the peptide may disrupt the structure of tumor-derived vesicles by forming pores by recognizing the lipid-based membrane with a high curvature of the tumor-derived vesicles and selectively binding thereto (see FIG. 12). Here, the “tumor-derived vesicles” are vesicles secreted from tumors, and have a diameter of 50 to 200 nm, 50 to 170 nm, 50 to 150 nm, 50 to 130 nm, 50 to 110 nm, 70 to 200 nm, 70 to 170 nm, 70 to 150 nm, 70 to 130 nm, 70 to 110 nm, 90 to 200 nm, 90 to 170 nm, 90 to 150 nm, 90 to 130 nm, 90 to 110 nm, 95 to 110 nm, 95 to 105 nm, 100 to 110 nm, or 100 nm, but the present invention is not limited thereto.

According to one example of the present invention, the peptide may be synthesized using a D-type amino acid, but the present invention is not limited thereto.

In another aspect of the present invention, the present invention provides a peptide, which is a peptide for cancer immunotherapy comprising an amino acid sequence of SEQ ID NO: 1 modified with a polyethylene glycol (PEG), and the PEG binds to the peptide via a linker.

In still another aspect of the present invention, the present invention provides a pharmaceutical composition for cancer immunotherapy, which comprises the peptide as an active ingredient.

In the present invention, the “cancer immunotherapy” may refer to the umbrella term for all systems that eliminate cancer cells by cancer-specific toxic immune cells (killer T cells) by inducing an immune response to a tumor-specific antigen or a tumor-associated antigen. For example, the method for inducing an immune response to a cancer antigen may use a gene, a protein, a virus, or dendritic cells.

In the present invention, the “immune checkpoint inhibitor” is a material that attacks cancer cells by the activation of T cells by blocking the activity of an immune checkpoint protein involved in T cell suppression, and may be, for example, one or more selected from the group consisting of PD-L1 inhibitors comprising atezolizumab, avelumab, or durvalumab, PD-1 inhibitors comprising pembrolizumab, nivolumab or spartalizumab, and CTLA-4 inhibitors comprising ipilimumab. According to one example or experimental example of the present invention, the immune checkpoint inhibitor may be an anti-PD-1 antibody, but the present invention is not limited thereto.

In yet another aspect of the present invention, the present invention provides a pharmaceutical composition for preventing or treating cancer, which comprises the peptide as an active ingredient.

The “cancer” used herein is the generic term for diseases caused by cells with an aggressive characteristic of dividing and growing cells by ignoring the normal growth limit, an invasive characteristic of penetrating into surrounding tissue, and a metastatic characteristic of spreading to other regions in the body. The cancer may be, for example, one or more selected from the group consisting of colorectal cancer, breast cancer, prostate cancer, melanoma, lung cancer, head and neck cancer, ovarian cancer, bladder cancer, stomach cancer, esophageal cancer, bile duct cancer, pancreatic cancer, liver cancer, cervical cancer, skin cancer, lymphoma, thyroid cancer, bone marrow cancer, endometrial cancer, and brain tumors, and according to one experimental example of the present invention, the cancer may be melanoma, lung cancer, skin cancer, or breast cancer, but the present invention is not limited thereto.

In yet another aspect of the present invention, the present invention provides a pharmaceutical composition for improving the sensitivity to cancer immunotherapy, which comprises the peptide as an active ingredient.

In the present invention, the “enhancing cancer immunotherapy sensitivity” means that, in cancer immunotherapy, the reaction sensitivity of cancer cells to a cancer immunotherapeutic agent such as an immune checkpoint inhibitor used for cancer immunotherapy is enhanced to improve the activity of cancer immunotherapy of a caner immunotherapeutic agent. In one experimental example of the present invention, it was confirmed that, in the co-administration of the peptide of the present invention with an immune checkpoint inhibitor, compared to the single treatment of an immune checkpoint inhibitor, a cancer immunotherapy effect is increased (see Experimental Example 7).

In addition, the present invention provides a pharmaceutical composition for inhibiting cancer metastasis, which comprises the peptide as an active ingredient. In the present invention, the pharmaceutical composition for cancer immunotherapy, the pharmaceutical composition for preventing or treating cancer, or the pharmaceutical composition for inhibiting cancer metastasis may further comprise an immune checkpoint inhibitor, in addition to the peptide. That is, through the co-administration of the peptide and the immune checkpoint inhibitor, cancer immunotherapy may be performed, but the present invention is not limited thereto.

In yet another aspect of the present invention, the present invention provides a pharmaceutical composition for cancer immunotherapy; preventing or treating cancer; or inhibiting cancer metastasis, which comprises the peptide represented by the amino acid sequence of SEQ ID NO: 1 and an immune checkpoint inhibitor as active ingredients.

In the present invention, the “cancer metastasis” refers to a condition in which a malignant tumor has spread to other tissues away from an organ where a malignant tumor occurs. As a malignant tumor that has started in one organ progresses, it spreads from the organ, which is the primary site to other tissues, and the spreading from the primary site to other tissues may be referred to as metastasis. Metastasis may be referred to as a phenomenon accompanying the progression of a malignant tumor, and as cancer cells are proliferated and cancer develops, metastasis may occur while acquiring new genetic traits. When cancer cells that have acquired new genetic traits invade a blood vessel and a lymphatic gland, circulate along the blood and a lymph, and eventually deposited and proliferated in other tissues, metastasis may occur. Even before cancer cells arrive, the frequency of metastasis or the growth of cancer tissue is influenced by a primary tumor by increasing the induction of circulating cancer cells through the formation of a region called “pre-metastatic niche.” This niche consists of Cd11b⁺ Gr-1⁺ myeloid cells recruited by lysyl oxidase (LOX) and S100A, and when the cells were inhibited, the formation of the niche is suppressed. In one experimental example of the present invention, it was confirmed that the peptide according to the present invention suppresses the formation of the pre-metastatic niche (see Experimental Example 8).

In yet another aspect of the present invention, the present invention provides a method of cancer immunotherapy or enhancing the sensitivity of cancer immunotherapy, which comprises administering a pharmaceutical composition comprising the peptide represented by the amino acid sequence of SEQ ID NO: 1 as an active ingredient into a subject in need thereof. Here, the pharmaceutical composition may further comprise an immune checkpoint inhibitor, and the method may further comprise administering an immune checkpoint inhibitor, separate from the pharmaceutical composition comprising the peptide.

In yet another aspect of the present invention, the present invention provides a method of cancer immunotherapy or enhancing the sensitivity of cancer immunotherapy, which comprises administering a peptide represented by the amino acid sequence of SEQ ID NO: 1 and an immune checkpoint inhibitor to a subject in need thereof.

In the present invention, there is no limitation in the order of administering the peptide and the immune checkpoint inhibitor, and the administration may be simultaneously, separately, or sequentially performed and may be performed once or more without limitation, but the present invention is not limited thereto.

In yet another aspect of the present invention, the present invention provides a use of the pharmaceutical composition comprising a peptide represented by the amino acid sequence of SEQ ID NO: 1 as an active ingredient for cancer immunotherapy or enhancing the sensitivity of cancer immunotherapy.

In yet another aspect of the present invention, the present invention provides a use of a peptide represented by the amino acid sequence of SEQ ID NO: 1 for preparing a drug for cancer immunotherapy or enhancing the sensitivity of cancer immunotherapy.

In yet another aspect of the present invention, the present invention provides a method of preventing or treating cancer, or inhibiting the metastasis of cancer, which comprises administering a pharmaceutical composition comprising a peptide represented by the amino acid sequence of SEQ ID NO: 1 as an active ingredient to a subject in need thereof.

In yet another aspect of the present invention, the present invention provides a use of a pharmaceutical composition comprising a peptide represented by the amino acid sequence of SEQ ID NO: 1 as an active ingredient for preventing or treating cancer, or inhibiting the metastasis of cancer.

In yet another aspect of the present invention, the present invention provides a use of a peptide represented by the amino acid sequence of SEQ ID NO: 1 for preparing a drug for preventing or treating cancer, or inhibiting the metastasis of cancer.

The pharmaceutical composition according to the present invention may further include a suitable carrier, excipient, and diluent which are commonly used in the preparation of pharmaceutical compositions. The excipient may be, for example, one or more selected from the group consisting of a diluent, a binder, a disintegrant, a lubricant, an adsorbent, a humectant, a film-coating material, and a controlled release additive.

The pharmaceutical composition according to the present invention may be used by being formulated, according to commonly used methods, into a form such as powders, granules, sustained-release-type granules, enteric granules, liquids, eye drops, elixirs, emulsions, suspensions, spirits, troches, aromatic water, lemonades, tablets, sustained-release-type tablets, enteric tablets, sublingual tablets, hard capsules, soft capsules, sustained-release-type capsules, enteric capsules, pills, tinctures, soft extracts, dry extracts, fluid extracts, injections, capsules, perfusates, or a preparation for external use, such as plasters, lotions, pastes, sprays, inhalants, patches, sterile injectable solutions, or aerosols. The preparation for external use may have a formulation such as creams, gels, patches, sprays, ointments, plasters, lotions, liniments, pastes, or cataplasmas.

As the carrier, the excipient, and the diluent that may be included in the pharmaceutical composition according to the present invention, lactose, dextrose, sucrose, oligosaccharides, sorbitol, mannitol, xylitol, erythritol, maltitol, starch, acacia rubber, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, and mineral oil may be used.

For formulation, commonly used diluents or excipients such as fillers, thickeners, binders, wetting agents, disintegrants, and surfactants are used.

As additives of tablets, powders, granules, capsules, pills, and troches according to the present invention, excipients such as corn starch, potato starch, wheat starch, lactose, white sugar, glucose, fructose, D-mannitol, precipitated calcium carbonate, synthetic aluminum silicate, dibasic calcium phosphate, calcium sulfate, sodium chloride, sodium hydrogen carbonate, purified lanolin, microcrystalline cellulose, dextrin, sodium alginate, methyl cellulose, sodium carboxymethylcellulose, kaolin, urea, colloidal silica gel, hydroxypropyl starch, hydroxypropyl methylcellulose (HPMC), HPMC 1928, HPMC 2208, HPMC 2906, HPMC 2910, propylene glycol, casein, calcium lactate, and Primojel®; and binders such as gelatin, Arabic gum, ethanol, agar powder, cellulose acetate phthalate, carboxymethylcellulose, calcium carboxymethylcellulose, glucose, purified water, sodium caseinate, glycerin, stearic acid, sodium carboxymethylcellulose, sodium methylcellulose, methylcellulose, microcrystalline cellulose, dextrin, hydroxycellulose, hydroxypropyl starch, hydroxymethylcellulose, purified shellac, starch, hydroxypropyl cellulose, hydroxypropyl methylcellulose, polyvinyl alcohol, and polyvinylpyrrolidone may be used, and disintegrants such as hydroxypropyl methylcellulose, corn starch, agar powder, methylcellulose, bentonite, hydroxypropyl starch, sodium carboxymethylcellulose, sodium alginate, calcium carboxymethylcellulose, calcium citrate, sodium lauryl sulfate, silicic anhydride, 1-hydroxypropylcellulose, dextran, ion-exchange resin, polyvinyl acetate, formaldehyde-treated casein and gelatin, alginic acid, amylose, guar gum, sodium bicarbonate, polyvinylpyrrolidone, calcium phosphate, gelled starch, Arabic gum, amylopectin, pectin, sodium polyphosphate, ethyl cellulose, white sugar, magnesium aluminum silicate, a di-sorbitol solution, and light anhydrous silicic acid; and lubricants such as calcium stearate, magnesium stearate, stearic acid, hydrogenated vegetable oil, talc, lycopodium powder, kaolin, Vaseline, sodium stearate, cacao butter, sodium salicylate, magnesium salicylate, polyethylene glycol (PEG) 4000, PEG 6000, liquid paraffin, hydrogenated soybean oil (Lubri wax), aluminum stearate, zinc stearate, sodium lauryl sulfate, magnesium oxide, Macrogol, synthetic aluminum silicate, silicic anhydride, higher fatty acids, higher alcohols, silicone oil, paraffin oil, polyethylene glycol fatty acid ether, starch, sodium chloride, sodium acetate, sodium oleate, dl-leucine, and light anhydrous silicic acid may be used.

As additives of liquids according to the present invention, water, dilute hydrochloric acid, dilute sulfuric acid, sodium citrate, monostearic acid sucrose, polyoxyethylene sorbitol fatty acid esters (twin esters), polyoxyethylene monoalkyl ethers, lanolin ethers, lanolin esters, acetic acid, hydrochloric acid, ammonia water, ammonium carbonate, potassium hydroxide, sodium hydroxide, prolamine, polyvinylpyrrolidone, ethylcellulose, and sodium carboxymethylcellulose may be used.

In syrups according to the present invention, a white sugar solution, other sugars or sweeteners, and the like may be used, and as necessary, a fragrance, a colorant, a preservative, a stabilizer, a suspending agent, an emulsifier, a viscous agent, or the like may be used.

In emulsions according to the present invention, purified water may be used, and as necessary, an emulsifier, a preservative, a stabilizer, a fragrance, or the like may be used.

In suspensions according to the present invention, suspending agents such as acacia, tragacanth, methylcellulose, carboxymethylcellulose, sodium carboxymethylcellulose, microcrystalline cellulose, sodium alginate, hydroxypropyl methylcellulose (HPMC), HPMC 1828, HPMC 2906, HPMC 2910, and the like may be used, and as necessary, a surfactant, a preservative, a stabilizer, a colorant, and a fragrance may be used.

Injections according to the present invention may include: solvents such as distilled water for injection, a 0.9% sodium chloride solution, Ringer's solution, a dextrose solution, a dextrose+sodium chloride solution, PEG, lactated Ringer's solution, ethanol, propylene glycol, non-volatile oil-sesame oil, cottonseed oil, peanut oil, soybean oil, corn oil, ethyl oleate, isopropyl myristate, and benzene benzoate; cosolvents such as sodium benzoate, sodium salicylate, sodium acetate, urea, urethane, monoethylacetamide, butazolidine, propylene glycol, the Tween series, amide nicotinate, hexamine, and dimethylacetamide; buffers such as weak acids and salts thereof (acetic acid and sodium acetate), weak bases and salts thereof (ammonia and ammonium acetate), organic compounds, proteins, albumin, peptone, and gums; isotonic agents such as sodium chloride; stabilizers such as sodium bisulfite (NaHSO₃) carbon dioxide gas, sodium metabisulfite (Na₂S₂O₅), sodium sulfite (Na₂SO₃), nitrogen gas (N₂), and ethylenediamine tetraacetic acid; sulfating agents such as 0.1% sodium bisulfide, sodium formaldehyde sulfoxylate, thiourea, disodium ethylenediaminetetraacetate, and acetone sodium bisulfite; a pain relief agent such as benzyl alcohol, chlorobutanol, procaine hydrochloride, glucose, and calcium gluconate; and suspending agents such as sodium CMC, sodium alginate, Tween 80, and aluminum monostearate.

In suppositories according to the present invention, bases such as cacao butter, lanolin, Witepsol, polyethylene glycol, glycerogelatin, methylcellulose, carboxymethylcellulose, a mixture of stearic acid and oleic acid, Subanal, cottonseed oil, peanut oil, palm oil, cacao butter+cholesterol, lecithin, lanette wax, glycerol monostearate, Tween or span, imhausen, monolan(propylene glycol monostearate), glycerin, Adeps solidus, buytyrum Tego-G, cebes Pharma 16, hexalide base 95, cotomar, Hydrokote SP, S-70-XXA, S-70-XX75(S-70-XX95), Hydrokote 25, Hydrokote 711, idropostal, massa estrarium (A, AS, B, C, D, E, I, T), masa-MF, masupol, masupol-15, neosuppostal-N, paramount-B, supposiro OSI, OSIX, A, B, C, D, H, L, suppository base IV types AB, B, A, BC, BBG, E, BGF, C, D, 299, suppostal N, Es, Wecoby W, R, S, M, Fs, and tegester triglyceride matter (TG-95, MA, 57) may be used.

Solid preparations for oral administration include tablets, pills, powders, granules, capsules, and the like, and such solid preparations are formulated by mixing the composition with at least one excipient, e.g., starch, calcium carbonate, sucrose, lactose, gelatin, and the like. In addition to simple excipients, lubricants such as magnesium stearate and talc are also used.

Examples of liquid preparations for oral administration include suspensions, liquids for internal use, emulsions, syrups, and the like, and these liquid preparations may include, in addition to simple commonly used diluents, such as water and liquid paraffin, various types of excipients, for example, a wetting agent, a sweetener, a fragrance, a preservative, and the like. Preparations for parenteral administration include an aqueous sterile solution, a non-aqueous solvent, a suspension, an emulsion, a freeze-dried preparation, and a suppository. Non-limiting examples of the non-aqueous solvent and the suspension include propylene glycol, polyethylene glycol, a vegetable oil such as olive oil, and an injectable ester such as ethyl oleate.

The pharmaceutical composition according to the present invention is administered in a pharmaceutically effective amount. In the present invention, “the pharmaceutically effective amount” refers to an amount sufficient to treat diseases at a reasonable benefit/risk ratio applicable to medical treatment, and an effective dosage level may be determined according to factors including types of diseases of patients, the severity of disease, the activity of drugs, sensitivity to drugs, administration time, administration route, excretion rate, treatment period, and simultaneously used drugs, and factors well known in other medical fields.

The composition according to the present invention may be administered as an individual therapeutic agent or in combination with other therapeutic agents, may be administered sequentially or simultaneously with therapeutic agents in the related art, and may be administered in a single dose or multiple doses. It is important to administer the composition in a minimum amount that can obtain the maximum effect without any side effects, in consideration of all the aforementioned factors, and this may be easily determined by those of ordinary skill in the art.

The pharmaceutical composition of the present invention may be administered to a subject via various routes. All administration methods can be predicted, and the pharmaceutical composition may be administered via, for example, oral administration, subcutaneous injection, intraperitoneal injection, intravenous injection, intramuscular injection, intrathecal (space around the spinal cord) injection, sublingual administration, administration via the buccal mucosa, intrarectal insertion, intravaginal insertion, ocular administration, intra-aural administration, intranasal administration, inhalation, spraying via the mouth or nose, transdermal administration, percutaneous administration, or the like.

The pharmaceutical composition of the present invention is determined depending on the type of a drug, which is an active ingredient, along with various related factors such as a disease to be treated, administration route, the age, gender, and body weight of a patient, and the severity of diseases.

As used herein, the “subject” refers to a subject in need of treatment of a disease, and more specifically, refers to a mammal such as a human or a non-human primate, a mouse, a rat, a dog, a cat, a horse, and a cow.

As used herein, the “administration” refers to providing a subject with a predetermined composition of the present invention by using an arbitrary appropriate method.

The term “prevention” as used herein means all actions that inhibit or delay the onset of a target disease. The term “treatment” as used herein means all actions that alleviate or beneficially change a target disease and abnormal metabolic symptoms caused thereby via administration of the pharmaceutical composition according to the present invention.

When the term “comprising or including” used herein is used, it means that other components may be further included rather than excluding other components unless otherwise stated.

In addition, the term “consisting of” used herein is considered to be a preferred embodiment of the term “comprising.”

Hereinafter, to help in understanding the present invention, exemplary examples will be suggested. However, the following examples are merely provided to more easily understand the present invention, and not to limit the present invention.

### EXAMPLES

### Example 1. Preparation of Peptides

An α-helical (AH-D) peptide (SGSWLRDVWDWICTVLTDFKTWLQSKL; SEQ ID NO: 1, including an amine group (—NH₂) at the C-terminus) and a control (NH-D) peptide (SGSWLRDDWDWECTVLTDDKTWLQSKL; SEQ ID NO: 2, including an amine group (—NH₂) at the C-terminus) were chemically synthesized using D-amino acids, and provided in a freeze-dried form with a purity of >90% (Anygen, Gwangju, South Korea). To prepare a peptide stock solution for an (in vitro) experiment, the freeze-dried peptide was first dissolved in dimethyl sulfoxide (DMSO), and then diluted in deionized water to a stock concentration of less than 2 mg/ml using 8 v/v % DMSO. The molar concentration of the peptide in the solution was determined by a UV-Vis spectroscopy (Optizen 3220UV, KLAB Co., Dajeon, Korea) experiment for measuring absorbance at 280 nm. A certain portion of the peptide stock solution was stored at −20° C. and thawed, followed by dilution in a suitable medium immediately before the experiment. In addition, for in vivo experiments, 3 mg of the AH-D peptide was dissolved in 20 μl of DMSO, and diluted with 980 μl of PBS immediately before administration into a mouse (50 μl per mouse; phosphate-buffered saline (PBS) containing 2% DMSO).

### Example 2. Cell Lines and Animal Model

Cell lines purchased from Korea Cell Line Bank (Seoul, Korea) were used unless specified otherwise. B16F10 murine melanoma and WM-266 human melanoma cell lines were cultured in Dulbecco's Modified Eagle Medium (DMEM; Capricorn Scientific, Ebsdorfergrund, Germany) containing 5% fetal bovine serum (FBS; Capricorn Scientific) and 1% penicillin/streptomycin (Capricorn Scientific). 4T1 murine mammary carcinoma (American Type Culture Collection, Manassas, Va.), MDA-MB-231 human mammary carcinoma, and NIH3T3 murine fibroblast cell lines were cultured in RPMI 1640 (Capricorn Scientific) containing 10% FBS and 1% penicillin/streptomycin. In addition, mouse tumor-associated fibroblasts (Cell Biologics Inc., Chicago, Ill.) were cultured in a complete fibroblast medium (Cell Biologics Inc.), and C166 murine endothelial cells (American Type Culture Collection) were cultured using an endothelial cell growth medium-2 kit (Promocell, Heidelberg, Germany). In all in vitro experiments, the cells were cultured in a 5% CO₂ humidified incubator at 37° C.

For an in vivo experiment, 5-6-week-old male C57BL/6 mice were purchased from Orient Bio Inc. (Seongnam, South Korea) and maintained in the absence of specific pathogens. All experiments involving live animals were performed in accordance with the relevant ethical regulations and protocols approved by the Institutional Animal Care and Use Committee (IACUC) of the Sungkyunkwan University.

### Example 3. Isolation and Analysis of Physical Properties of Tumor-Derived Exosomes (T-EXO)

T-EXO was isolated from an in vitro cell culture medium using a tangential flow filtration (TFF) system with an Omega™ membrane filter capsule (Pall Corporation, Port Washington, N.Y.) having a molecular weight cutoff of 300 kDa by slight changing a previously reported method (Woo C H et al., Journal of Extracellular Vesicles 2020, 9(1): 1735249). That is, first, B16F10 cells, which are a melanoma cell line, were washed twice with PBS, and cultured in serum-free DMEM for 24 hours, a cell culture supernatant was collected and centrifuged (2,000 g, 20 min), and cell debris and large vesicles were removed by filtration through a 0.22-μm filter. For an in vivo experiment, cell-free plasma was obtained from a blood sample by a method reported in previous research (Chen G et al., Nature 2018, 560(7718): 382-386) to isolate T-EXO from mouse plasma. The mouse plasma-derived T-EXO was subsequently purified using a total exosome isolation kit (Invitrogen, Carlsbad, Calif.) according to the manufacturer's instructions. In addition, in order to extract tumor-derived exosomes from a mouse tumor model, exosomes were isolated using ultracentrifugation (100,000 g, 90 min) according to a method reported in previous research (Son et al., Biomaterials 2021, 276: 121058).

To measure T-EXO size distribution, a T-EXO suspension (PBS containing 2×10⁹ particles/ml) was analyzed using a nanoparticle tracking analysis system (NTA; Nano Sight LM10, Malvern Instruments, UK). The time-resolved Brownian motion of the particles was measured by repeating the test three times for 30 seconds, and thereby the size distribution of the particles was measured. In addition, to confirm T-EXO surface PD-L1 expression and an exosome-specific biomarker, Western blotting was performed. Cell and exosome lysates were extracted using a radio-immunoprecipitation assay buffer solution, isolated by size through 12% sodium dodecyl sulfate (SDS) polyacrylamide gel electrophoresis, and blotted on a polyvinylidene difluoride membrane (Millipore, Burlington, Mass., U.S.A.). Staining was carried out at 4° C. overnight using anti-CD9 (Clone EPR2949, Abcam), anti-PD-L1 (Clone EPR 20529, Abcam), anti-GM130 (Clone B-10, SCBT), and anti-β-actin (Clone B31.15, Sigma-Aldrich) primary antibodies. After washing with a 0.1% Tween-containing Tris buffer solution, staining was carried out at room temperature for 1 hour using a horseradish peroxidase-conjugated secondary antibody, and chemiluminescence of the secondary antibody-stained portion was imaged using a chemiluminescent substrate solution.

### Example 4. Synthesis of Tumor-Responsive PEG-Linker and Synthesis of Tumor-Responsive Peptide

The synthesis of polyethylene glycol (PEG) and a pH-responsive linker (3-(4-methyl-2,5-dioxo-2,5-dihydrofuran-3-yl)propanoic acid, Ambeed, Ill.) was attempted. First, after drying the linker and the PEG overnight under low pressure, 2 ml of dichloromethane (DCM) was added and dissolved under nitrogen. Subsequently, while adding 46.3 μl of oxalyl chloride and 8 μl of N,N-dimethylformamide, the resulting solution was maintained at 0° C. for 10 minutes and then stirred at room temperature for 1 hour. Afterward, a DCM solvent in the linker solution was evaporated under reduced pressure, 4 ml of a PEG solution (45 mg/ml, DCM) and 6 μl of pyridine were added to perform a reaction. After one night, the reaction was stopped by adding 10 ml of a saturated aqueous solution of ammonium chloride, and only the debris was collected separately using a separatory funnel and precipitated in diethyl ether. Then, only the precipitate was collected by centrifugation (2500 g, 15 min), and dried using a vacuum oven for 24 hours. The linker was bound to an amine group of the peptide to form a carboxy-dimethylmaleic amide (CDM) bond, and for PEG-linker-modified peptide (PEG-CDM-pep) synthesis, an AH peptide and a 7.5-fold excess of PEG-linker were reacted in PBS for 4 hours, and to remove unreacted peptides and PEG-linkers, dialysis was performed in pH 7.4 PBS using a membrane with a 7 kDa cutoff. The synthesis of PEG-modified peptide (PEG-pep) used as a control was performed through the same reaction as described above using PEG to which a succinimidyl glutarate ester functional group was bound.

As the peptide prepared in Example 1 has an additional amine (—NH₂) group at the C-terminus, amine groups are present at both termini, in addition to the amine (—NH₂) group at the N-terminus. The PEG-linker has reactivity with an amine, and the reaction proceeded at both termini of the peptide.

### Example 5. Characterization of PEG-Linker-Modified Peptide

To confirm the binding between the linker and the PEG, each of the linker, PEG, and PEG-linker was dissolved in CDCl₃ and subjected to ¹H-NMR measurement (500 MHz, Bruker, Mass.). In addition, circular dichroism spectroscopy was used to determine whether the α-helical secondary structure was maintained after PEG modification of the peptide.

### Example 6. Evaluation of pH Sensitivity of PEG-Linker-Modified Peptide

To confirm the pH sensitivity of the tumor-sensitive linker, a release behavior evaluation was carried out. First, a maleimide-conjugated cy5.5 phosphor was bound to the thiol functional group of the cysteine portion of a peptide. To this end, after dissolving the AH peptide in a PBS (pH 7.4), a 100-fold excess of tris(2-carboxyethyl)phosphine (TCEP) was added to react at room temperature for 20 minutes, and then a single peptide was formed by the oxidation of a disulfide bond. Afterward, after removing TCEP using a 2 kDa cutoff dialysis membrane, a 10-fold excess of phosphor was added and reacted at room temperature for 24 hours. After the reaction, an unreacted phosphor was removed using a 2 kDa cutoff dialysis membrane, and then binding to the PEG was performed in the above-mentioned manner.

The peptide release behavior was evaluated using a 7 kDa cutoff membrane. Each peptide was dissolved in each PBS solution (pH 7.4 or 6.5), and the pH sensitivity of the peptide was evaluated through the absorbance measurement of the phosphor binding to the single peptide released from the membrane. The absorbance at a single wavelength of 675 nm was measured using UV-vis spectroscopy.

### Example 7. Evaluation of In Vitro T-EXO Disruption Activity

The T-EXO number before and after peptide treatment was measured using a nanoparticle tracking analysis system (NTA; Nano Sight LM10, Malvern Instruments, UK). A T-EXO suspension (PBS containing 1×10⁹ particles/ml) was treated under the indicated experimental conditions at room temperature in the presence or absence of the peptide. After treatment, the suspension was centrifuged at a designated time point (2,000 g, 15 min), and an aggregate was removed before loading into a syringe pump system. To evaluate the disruption effect of the peptide according to pH, T-EXO was dispersed in PBS at a specific pH (pH 7.4, 6.8 or 6.5) and then treated with or without the peptide for 5 minutes. The time-resolved Brownian motion of each particle was recorded for 30 seconds, repeated three times per sample. NTA 3.1 software was used for the control of experimental parameters of recorder video and data analysis. The relative concentration of T-EXO was calculated by dividing a particle concentration at each measurement time point by the initial particle concentration before treatment.

### Example 8. Evaluation of In Vitro T-EXO Disruption Activity According to pH

The T-EXO number before and after peptide treatment was measured using a nanoparticle tracking analysis system. To evaluate PEG-linker-modified peptide activity according to pH, after the peptide was left at room temperature for 3 hours in PBS of a specific pH (pH 7.4 or 6.5), the same amount of T-EXO was injected into each test tube and left for 10 minutes. Then, an aggregate was removed through centrifugation (2,000 g, 15 min), and a T-EXO residual concentration was measured. The relative concentration of T-EXO was calculated by dividing by the concentration of the injected T-EXO.

### Example 9. Evaluation of Liposome Disruption Activity

To characterize the kinetics of peptide-liposome interactions using the Q-Sense E4 instrument (Biolin Scientific AB, Gothenburg, Sweden), quartz crystal microbalance-dissipation (QCM-D) was measured. First, small monolayer liposomes consisting of a 1,2-dioleoyl-sn-glycero-3-phosphocholine (DOPC) lipid were prepared by extrusion in 10 mM Tris buffer (pH 7.4) with 150 mM NaCl, and confirmed by dynamic light scattering measurement to have a diameter of less than 70 nm. Before the experiment, liposomes were diluted to a bulk lipid concentration of 0.125 mg/ml using a suitable buffer [10 mM Tris buffer (pH 7.4), 10 mM Bis-Tris buffer (pH 6.8) or 10 mM Bis-Tris buffer (pH 6.5); all of the buffers were used with 150 mM NaCl], and the same buffer was used for both the QCM-D experimental step and the peptide dilution step.

In the case of the QCM-D experiment, by monitoring the time-dependent shift of resonance frequency (Δf) and energy dissipation (ΔD) signals, an adsorption process for characterizing liposome adsorption and peptide-liposome interactions was tracked. For all experiments, a QCM-D sensor chip (Biolin Scientific) coated with titanium oxide was used, under appropriate pH conditions (pH 7.4, 6.8 or 6.5), 8, 16, 24, or 32 μM of the peptide was injected under continuous flow at a volume rate of 50 μl/min, and liposomes were first adsorbed to the sensor surface prior to injection. Experimental data was collected using the QSoft (Biolin Scientific) software program, and QTools (Biolin Scientific) and OriginPro 8.5 (OriginLab) software programs were used for data processing. The indicated data is transmitted at the fifth overtone, and the maximum Δf and ΔD shifts correspond to the inflection points of peptide-induced liposome rupture kinetics. The time scale for the peptide-induced liposome rupture was calculated based on the initial time point at which a change in the Δf signal was caused by peptide addition until the Δf signal returns to a value of −60 Hz relative to the measurement baseline only in a buffer solution.

### Example 10. Interfacial Hydrophobicity Modeling

To calculate interfacial hydrophobicity, the membrane protein explorer (MPEx) Java program (ver. 3.3.0, https://blanco.biomol.uci.edu/mpex/) was used. In this program, a Totalizer mode was used, the amino acid sequence of an amidated AH-D peptide was inserted to calculate the Gibbs free energy of water-bilayer partitioning based on the Wimley-White interfacial hydrophobicity scale. Calculation was performed by treatment with charged or uncharged aspartic acid (Asp) residues, and for pH-dependent calculation, the pKa value of the Asp residue in the corresponding peptide was 7.7, and it was assumed that the fractions of charged Asp residues and uncharged Asp residues in the peptide at pH 7.4, 6.8, or 6.5 are estimated by the Henderson-Hasselbalch equation. The corresponding calculation was made by assuming that a peptide mixture of charged or uncharged Asp residues was present at each pH condition, and accounted for the partial contribution of each peptide species to membrane partitioning.

### Example 11. PD-1/PD-L1 Binding Analysis

The amounts of PD-L1)(PD-L1^{EXO} in plasma exosomes isolated from wild-type (WT) and B16F10 melanoma tumor-bearing mice were quantified using a mouse PD-L1 enzyme-linked immunosorbent assay (ELISA) kit (DY1019-05, R&D Systems, Minneapolis, Minn.) according to the manufacturer's protocol. In addition, the binding ability of the PD-L1^{EXO} to PD-1 was confirmed by ELISA. All steps of PD-1 binding analysis were carried out at room temperature, and EXO containing the same amount of protein or T-EXO (100 μl per well) was treated with 3 μM of the peptide or without the peptide for 10 minutes, and then a sample was added to a PD-L1 antibody-coated 96 well plate. After treating the plate for 2 hours, the plate was washed with PBS-T (PBS containing 0.05% Tween-20). Subsequently, 100 μl of a biotin-labeled murine PD-1 protein (4 μg/ml; cat no. 71118, BPS Bioscience, San Diego, Calif.) was added to the plate and treated for 2 hours. Wells were washed with PBS-T three times, and treated with horseradish peroxidase-conjugated streptavidin (100 μl per well; R&D Systems) diluted in 0.5% BSA-containing PBS for 30 minutes. After washing with PBS-T, the plate was treated with tetramethylbenzidine (R&D Systems) and the reaction was stopped by adding 0.5 N H₂SO₄. The binding ability of PD-L1^{EXO} to PD-1 may be quantified by measuring an optical density at 450 nm using a microplate reader (BioTek Instruments, Winooski, Vt.).

### Example 12. Analysis of PD-1/PD-L1 Binding According to pH

The amount of PD-L1 in plasma exosomes isolated from wild-type (WT) and B16F10 melanoma tumor-bearing mice was assessed using a mouse-derived PD-L1 ELISA kit (DY1019-05, R&D system, Minneapolis, Minn.) according to the manufacturer's instructions. In addition, to analyze the PD-1/PD-L1 binding inhibitory effect of a tumor-responsive peptide, ELISA was used. Each peptide was dispersed in PBS of a specific pH (pH 7.4 or 6.5) and left for one hour, followed by injection of the same amount of EXO or T-EXO and a reaction for 10 minutes. Two hours after the injection of the prepared sample into a PD-L1 antibody-coated 96-well plate, the plate was washed with PBS-T (PBS containing 0.05% Tween-20). Afterward, a 100 μl biotin-labeled PD-1 protein (4 μg/ml; cat no. 71118, BPS Bioscience, San Diego, Calif.) was added to the plate, and treated for 2 hours. After washing each well with PBS-T, horseradish peroxidase-conjugated streptavidin was injected, and the plate was left for 30 minutes. After washing, tetramethylbenzidine (R&D Systems) was injected, a 0.5 N sulfuric acid aqueous solution was added to stop the reaction. PD-1/PD-L1 binding ability was assessed by measuring an optical density at 450 nm using a microplate reader (BioTek Instruments, Winooski, Vt.).

### Example 13. Isolation and Stimulation of CD8+ T Cells

The spleen was obtained from a C57BL/6 mouse and passed through a 40-μm cell strainer, thereby obtaining a single cell suspension, and then red blood cells were removed using a red blood cell (RBC) lysis buffer (BioLegend, San Diego, Calif.) containing NH₄Cl, K₂CO₃ and ethylene-diamine-tetraacetic acid (EDTA). CD8⁺ T cells were extracted through magnetic isolation using a mouse naïve CD8a⁺ T cell isolation kit (Miltenyi Biotec, Bergisch Gladbach, Germany) and an LS column (Miltenyi Biotec) according to the manufacturers' instructions. The prepared naïve CD8⁺ T cells were seeded, treated with a CD3 antibody (5 μg/ml) and a CD28 antibody (2 μg/ml) in a T cell culture medium (a RPMI1640 medium supplemented with a 1× non-essential amino acid solution (M7145), 50 μM β-mercaptoethanol (60-24-2), and 100 IU/ml IL-2 (SRP3242; Sigma), and cultured for 24 hours to activate T cells.

### Example 14. Cellular Uptake of T-EXO by T Cells

The splenic CD8⁺ T cells were prepared by the method of Example 13 and activated, and then 1×10⁶ cells were seeded in each well of a 12-well plate in which a glass surface was coated with fibronectin (2 μg/cm²; F1141, Sigma). Subsequently, Cy5.5-labeled T-EXO (100 μg protein/me was pre-treated for 10 minutes with/without a 3 μM peptide and added to each well. The cells were cultured for 1, 3, or 6 hours with a T-EXO sample, and then rinsed with PBS three times. Afterward, the cells were fixed to a glass coverslip with 4% paraformaldehyde, and rinsed with PBS three times. To visualize the CD8⁺ T cells, the coverslip was mounted on the glass slide, and the cell nucleus was stained using a DAPI Fluoromount-G stain (Southern Biotech, Birmingham, Ala.). The slide was imaged using a Leica TCS SP8 confocal microscope (Leica Microsystems, IL, U.S.A.), and the fluorescence intensity was quantified using a Leica LAS-X software program (Leica Microsystems).

### Example 15. Cellular Uptake of T-EXO by T Cells According to pH

CD8⁺ T cells were prepared by the method of Example 13, and 3×10⁵ cells were seeded in a culture plate for a confocal microscope, in which the glass surface was coated with fibronectin (2 μg/cm²; F1141, Sigma). Subsequently, the cells were left for 10 minutes with a 2 μM peptide pretreated with DIO phosphor-labeled T-EXO (100 μg protein/ml) at different pHs, and added to each culture plate. Three hours later, the cells were fixed with 4% paraformaldehyde, and washed with PBS three times. And then, the Hoechst stain was used to stain the cell nucleus, and the nucleus was imaged using a confocal microscope (Leica Microsystems, IL, U.S.A.).

To quantify the cellular uptake tendency of T-EXO, activation was carried out in a 24-well plate (2×10⁶ cells per well) in the manner described in Example 13. Subsequently, the cells were left for 10 minutes with a 2 μM peptide pretreated with a DIO phosphor-labeled T-EXO (100 μg protein/ml) at different pHs, and added to each well. After three hours, the emission intensity of the DIO phosphor was measured using a flow cytometer (Guava EasyCyte, Millipore, Burlington, Mass.).

### Example 16. Cytotoxicity Assay

To assess the cytotoxicity of an AH peptide, PEG-modified peptide and PEG-linker-modified peptide, a cytotoxicity assay (3-(4,5-dimethylthiazol-2-yl)-2,5-diphenyltetrzolium bromide (MTT) assay) for the melanoma cell line B16F10 and a fibroblast cell line NIH3T3 of mouse was carried out. First, 10⁴ cells of each cell line were seeded in a 96-well plate, and cultured for 24 hours. After culturing for 4 hours in a serum-free culture medium, the peptides at each concentration were injected into the wells and further incubated for 4 hours. After discarding a supernatant, 0.5 mg/ml of MTT-containing medium was treated and then left for 2 hours, formazan crystals in the cells were dissolved in each well using dimethyl sulfoxide. Then, cytotoxicity was analyzed by measuring absorbance at 570 nm using a microplate reader.

### Example 17. In Vitro T Cell Analysis

T-EXO (200 μg protein/mi) was pretreated for 10 minutes with/without a 6 μM peptide, and the produced mixture is described below as a T-EXO suspension.

To investigate a T-EXO-mediated T cell functional impairment effect and a PEG-linker-modified peptide-mediated decrease in T-EXO functional impairment, CD8⁺ T cells were isolated from the spleen and activated by the method of Example 13. For cell proliferation analysis using carboxyfluorescein succinimidyl ester (CFSE), cells were stained with CFSE (5 μM; CellTrace CFSE cell proliferation kit, Thermo Fisher Scientific, Waltham, Mass.) before stimulation with an anti-CD3 antibody (Clone 145-2c11, BioLegend) and an anti-CD28 (Clone 37.51, BioLegend) antibody. That is, staining was carried out at 37° C. for 10 minutes in a serum-free medium, and addition of a volume of low-temperature medium (10% FBS) equal to 5 times the cell suspension to stop the staining. Afterward, the CFSE-stained cells were rinsed twice with a fresh medium and seeded in a 24-well plate (2×10⁶ cells per well), followed by stimulation for 24 hours. After incubation with the T-EXO suspension for 4 days, the harvested cells were rinsed with 1% FBS-containing PBS three times, and further analyzed using flow cytometry (Guava EasyCyte, Millipore, Burlington, Mass.). Among the CFSE-labeled cells, T-EXO-free, stimulated cells and unstimulated cells were selected as positive and negative (non-dividing) controls, respectively. The supernatant was collected and the production of cytokines (IFN-γ and TNF-α) was quantified using an ELISA kit (R&D Systems) according to the manufacturer's protocol.

In addition, to detect intracellular Ki-67 expression, the isolated CD8⁺ T cells were cultured with the T-EXO suspension for 48 hours. Afterward, 2-3×10⁶ cells were agitated at 350 g for 5 minutes and treated with an ice-cold 70% ethanol solution according to the manufacturer's protocol. After culturing at −20° C. for 1 hour, the cells were washed with 1% FBS-containing PBS twice, and 1×10⁶ cells were stained with anti-mouse Ki67-PE/Dazzle™ 594 (Clone 16A8, BioLegend) for 30 minutes, washed twice, and analyzed by flow cytometry.

In addition, for intracellular Grz-B staining, the isolated CD8⁺ T cells were cultured with a T-EXO suspension for 48 hours and treated for further 6 hours with monensin (1 μg/ml; Thermo Fisher Scientific). After treatment, 1×10⁶ cells were collected and treated with an anti-mouse CD16/32 antibody (Clone 2.4G2, BioLegend), and then left on ice for 15 minutes to block the reaction. Subsequently, the cells were labeled with anti-mouse CD8-FITC (Clone 53-6.7, BD Biosciences, San Jose, Calif.) in a dark room at 4° C. for 30 minutes, and washed with 1% FBS-containing PBS twice. Afterward, the surface-stained cells were fixed and permeabilized with Cytofix/Cytoperm buffer (BD Biosciences), and washed twice with 1× Perm/Wash buffer (BD Biosciences). After staining with anti-human/mouse Granzyme B-PE (Clone QA16A02, BioLegend), the cells were analyzed through flow cytometry.

### Example 18. Evaluation of In Vivo Antitumor Efficacy

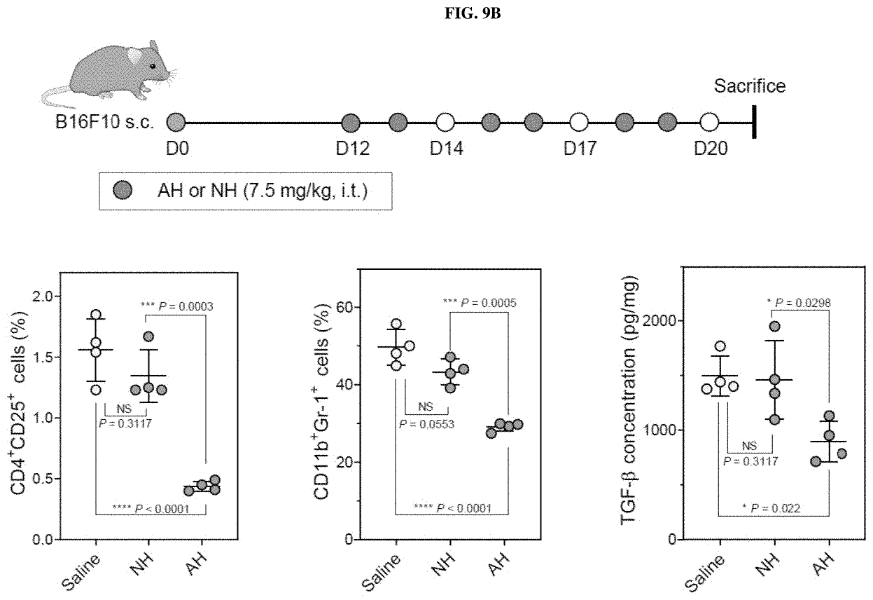

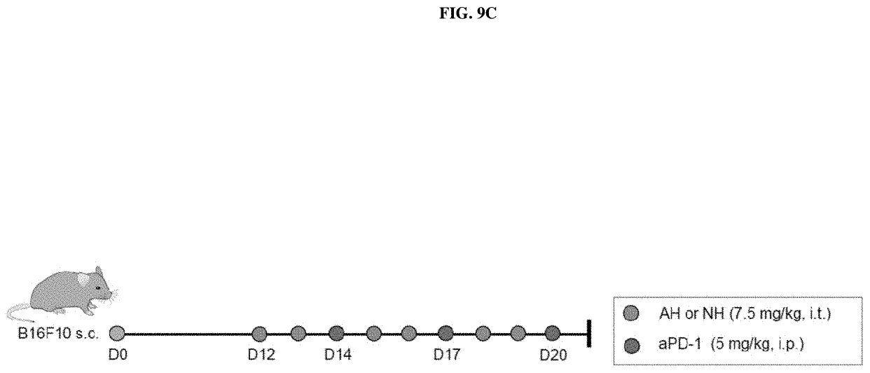

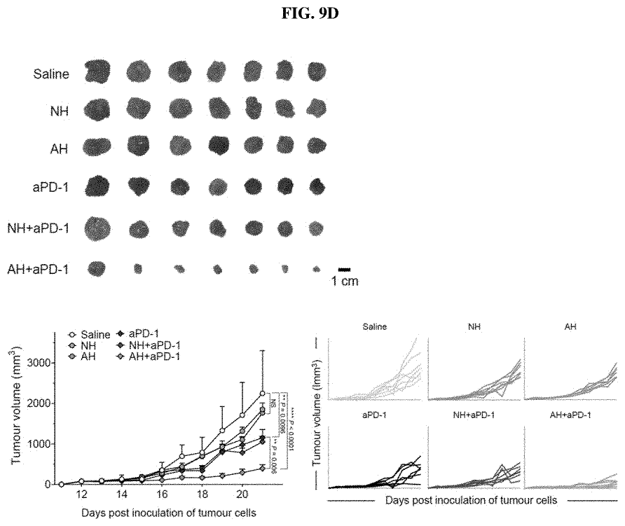

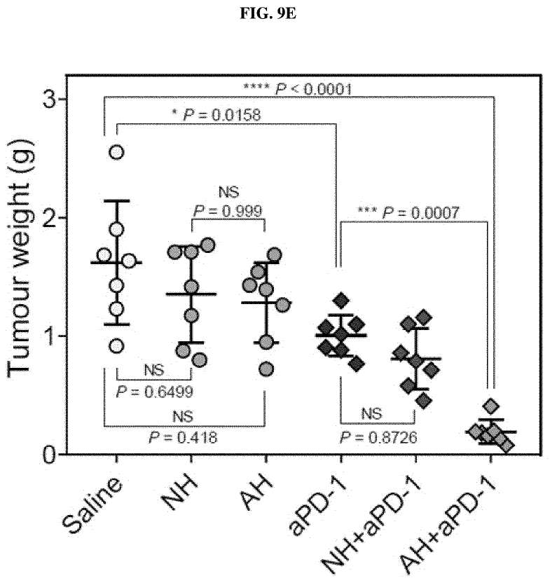

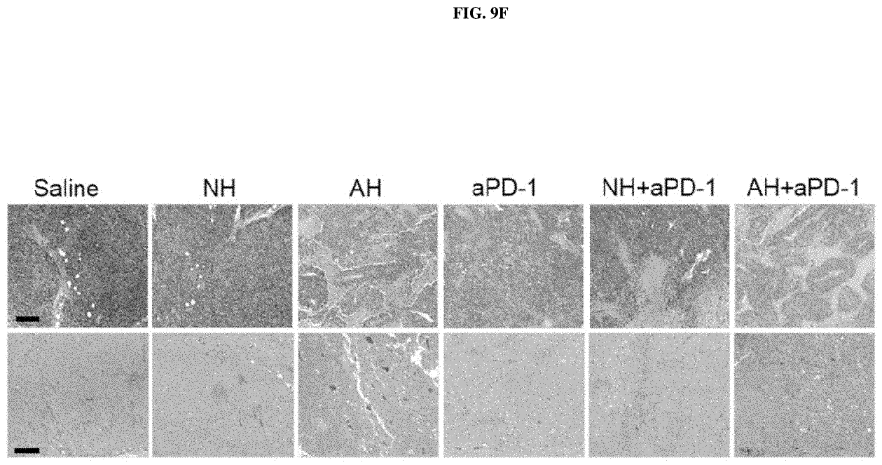

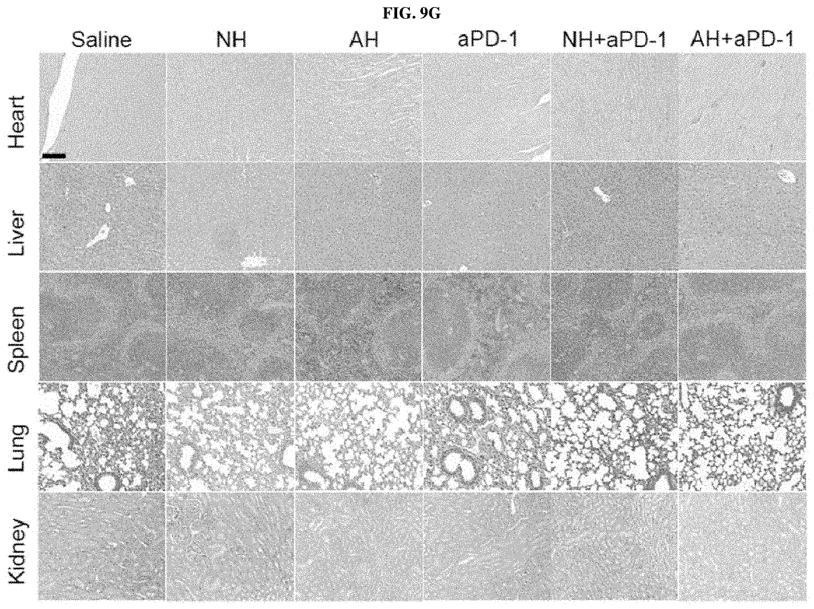

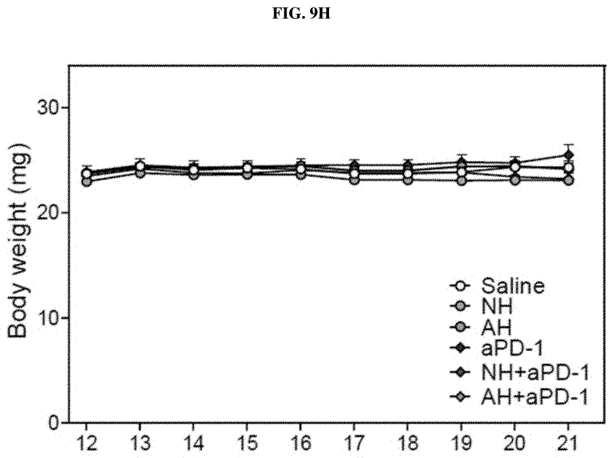

To prepare tumor-bearing mice, 1×10⁶ melanoma B16F10 cells were suspended in 100 μl of PBS, and subcutaneously injected into the right flank of a C57BL/6 mouse. On day 12 after injection, the B16F10 tumor-bearing mice were randomly divided into six treatment groups (n=7 mice per group): saline, NH-D peptide (NH), AH-D peptide (AH), aPD-1 antibody (Clone RMP1-14, Catalog BE0146, InVivoMAb), NH+aPD-1, and AH+aPD-1. 50 μl of 7.5 mg/kg peptide was injected into a tumor of each mouse, and 100 μl of 5 mg/kg aPD-1 antibody was intraperitoneally administered. The peptide was treated twice before the start of antibody treatment, and the dosing schedule is shown in FIG. 9C. Tumor volumes and body weights were recorded daily, and calculated as follows: Volume (mm³)=0.5×length (mm)×width (mm)×width (mm).

On day 21, the mice were euthanized to collect tumor tissues, related organs and blood samples for further analysis.

### Example 19. Flow Cytometry

The dissociation of the harvested B16F10 tumors was performed using a mouse tumor dissociation kit (Miltenyi Biotec) and a Gentle MACS dissociator (Miltenyi Biotec) according to the manufacturers' instructions. A tumor homogenate was sequentially passed through 70- and 40-μm cell strainers to obtain a single cell suspension, followed by RBC lysis. Additionally, the cells were magnetically sorted using a Miltenyi CD45 TIL microbead and a CD11b microbead to analyze T cell and bone marrow cell populations in tumors. Subsequently, 2×10⁶ sorted cells were blocked with anti-mouse CD16/32 antibodies on ice for 15 minutes, and fluorescence staining was performed using an antibody in a dark room at 4° C. for 30 minutes: anti-CD3-FITC (Clone 145-2C11, BioLegend), anti-CD8-PE/Dazzle™ 594 (Clone 53-6.7, BioLegend), anti-CD4-FITC (Clone GK1.5, BioLegend), anti-CD25-PE (Clone PC61, BioLegend), anti-CD11b-FITC (Clone M1/70, BioLegend), and anti-Ly-6G/Ly-6C-PE (Gr-1; Clone RB6-8-05, BioLegend).

Afterward, the populations of CD3⁺ CD8⁺ T cells, CD4⁺ CD25⁺ regulatory T cells (Tregs) and CD11b⁺Gr-1⁺ bone marrow-derived suppressor cells (MDSC) were analyzed through flow cytometry (Guava EasyCyte). To analyze the proliferation of splenic T cells and an effector function, the splenic cells were inoculated into a 24-well plate at a density of 1×10⁶ cells with a T cell culture medium. The cells were stimulated for 48 hours in the presence of 100 IU/ml IL-2 and a tumor lysate. Experimental procedures for CFSE analysis and staining with anti-Grz-B-PE were carried out as described above.

### Example 20. In Vivo Anti-Metastasis Test

To simulate T-EXO-mediated pre-metastatic niche formation, C57BL/6 mice were pretreated with T-EXO (100 μg protein) alone or in combination with a 3 μM peptide on day 0, 2 or 4 for 10 minutes, and injected intravenously. In the case of a control not treated with T-EXO, the same volume (200 μl) of saline was intravenously injected into the mouse. On day 6 after injection, a total of 2×10⁵ B16F10 cells were administered into the mice through the lateral caudal vein. After 4 weeks, the mice were sacrificed to obtain the lungs and the livers. The collected organs were fixed with 10% neutral buffered formalin and further processed for histological examination. Metastatic lesions were analyzed by hematoxylin and eosin (H&E) staining and immunofluorescence staining.

### Example 21. Immunofluorescence Staining

Immunofluorescence staining was performed on a frozen tissue section or a paraffin-embedded tissue section. The frozen tissue section was fixed with ice-cold acetone, and blocked with 1% BSA-containing PBS for 1 hour. The paraffin-embedded tissue section was steamed with citrate buffer (pH 6.0) to perform an antigen retrieval step, and blocked with 1% BSA-containing PBS for 1 hour. A primary antibody was diluted with a staining buffer (PBS containing 1% BSA and 0.1% Triton X-100) according to the manufacturer's instructions, and the sections were cultured overnight with a primary antibody at 4° C., and washed with PBS-T three times. The sections were further stained with a secondary antibody at room temperature for 1 hour except for a phosphor-conjugated primary antibody. The used antibodies are as follows: anti-CD8-Alexa Fluor 594 (Clone 53-6.7, BioLegend), anti-a-SMA (Clone 1A4, Invitrogen), anti-fibronectin (cat no. ab2413, Abcam, Cambridge, UK), anti-CD31 (PAS-16301, Invitrogen), anti-S100A4 (Clone S100A4, BioLegend), goat anti-rabbit IgG-Alexa Fluor 594 (cat no. ab150080, Abcam). The nucleus was stained with DAPI Fluoromount-G, and then its image was obtained using a confocal microscope.

### Example 22. Evaluation of Blood Exosome Inhibition of PEG Modified Peptide

To prepare a tumor mouse model, 1×10⁶ melanoma B16F10 cells were suspended in 100 μl PBS and injected subcutaneously into the right limb of a C57BL/6 mouse. The tumor mouse models were randomly divided into five treatment groups (n=3 mice per group): saline, 40 mg/kg of AH peptide, 10, 20, or 40 mg/kg of PEG-linker-modified peptide. A 200 μl of sample was intravenously injected into each mouse twice, exosomes contained in the tumor were isolated by the method described in Example 3, and the amount of PD-L1 expressed on the exosome surface was assessed by ELISA. Afterward, relative to the total protein amount of the exosomes isolated from the tumor tissue, a PD-L1 expression level was calculated, showing the relative proportion of the tumor-derived exosomes.

### Example 23. Statistical Analysis

All data was presented as mean±SD. Statistical comparison was evaluated using one-way analysis of variance (ANOVA), and significant difference between data sets are expressed as follows: *p<0.05, **p<0.01, ***p<0.001 and NS; not significant.

### EXPERIMENTAL EXAMPLE

### Experimental Example 1. T-EXO Disruption Effect of Peptide

1-1. Confirmation of Biophysical Characteristics of Peptide

To implement a lipid envelope exosome disruption (LEED) strategy, a high curvature lipid membrane (diameter <300 nm) was disrupted, and the use of a 27-mer amphipathic, α-helical (AH-D) peptide that can suppress a membrane-enclosed virus in vivo was changed. The LEED strategy is shown schematically in FIG. 12. A control (NH-D) peptide was also tested, which is a mutant version of the AH-D peptide with a random coil secondary structure and having no membrane disruption. The biophysical characteristics of the two peptides were confirmed by circular dichroism spectroscopy and quartz crystal microbalance-dissipation (QCM-D), which characterize the secondary structure and membrane disruption activity of each peptide.

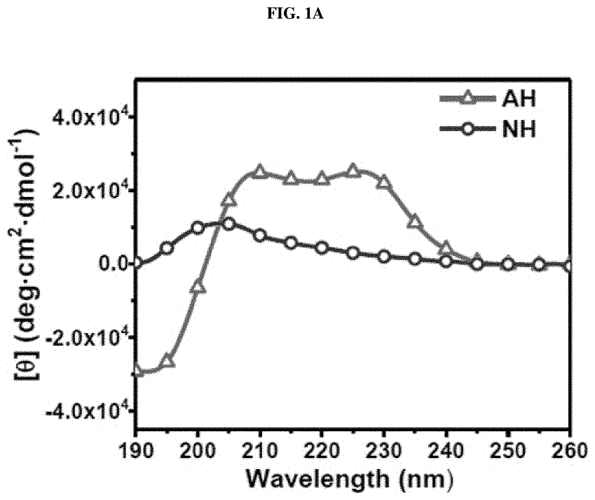

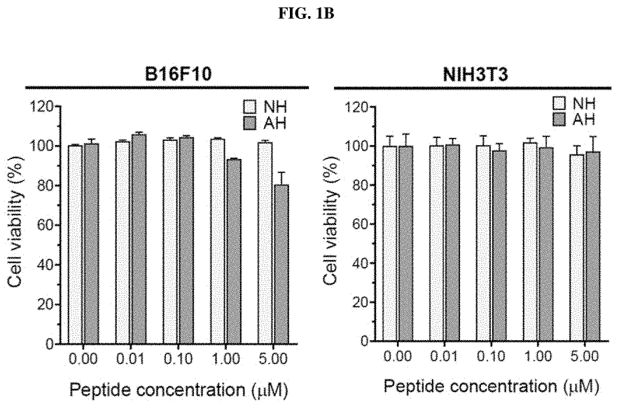

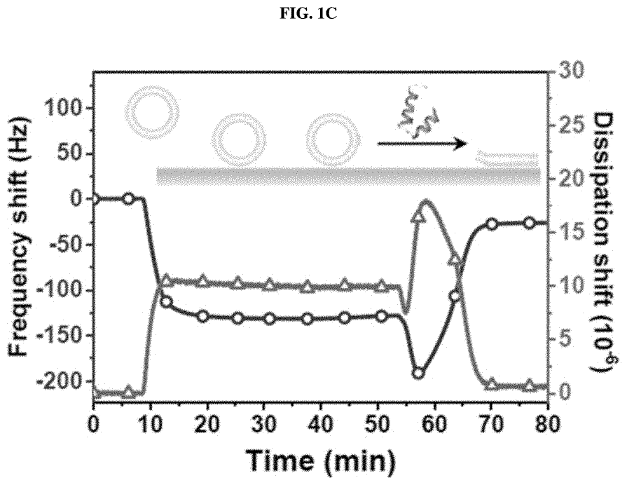

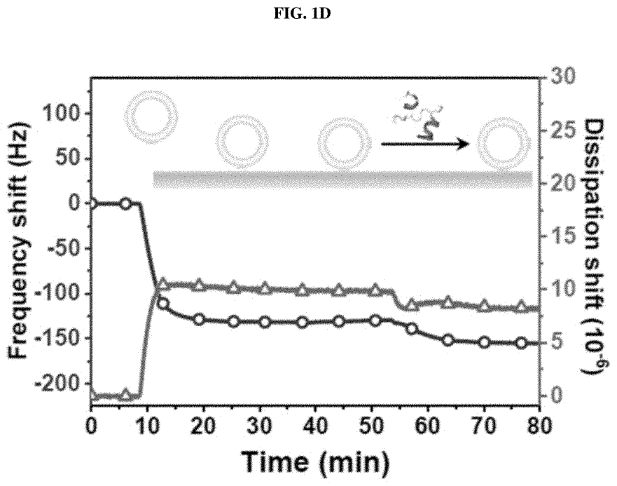

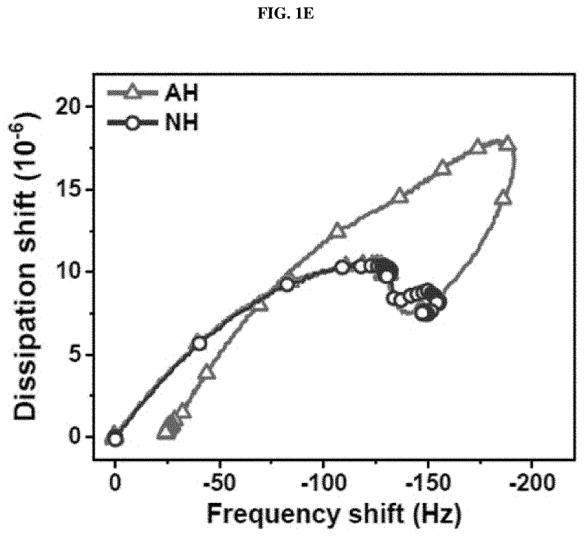

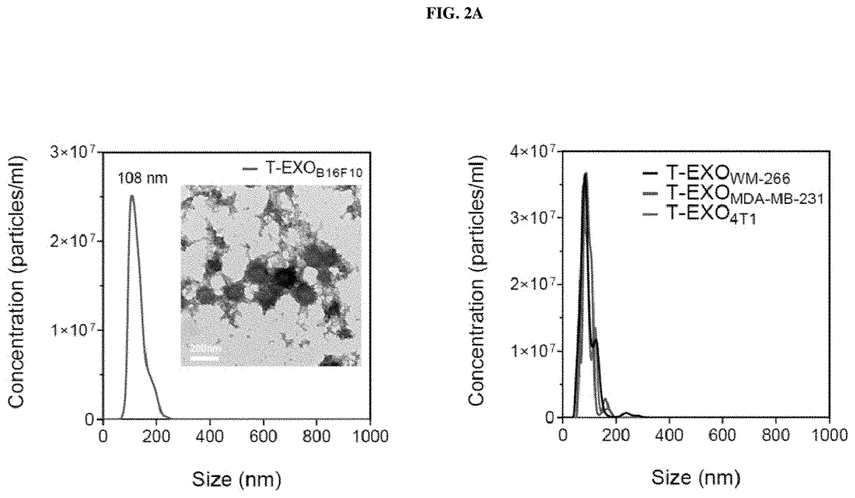

FIG. 1A shows that the AH-D peptide (indicated by a red triangle) and the NH-D peptide (indicated by a blue circle) mainly have α-helical and random coil secondary structures, respectively, through circular dichroism spectroscopy (n=3).

FIG. 1B shows that, as a result of confirming cytotoxicity in mouse melanoma B16F10 cells and a normal cell line NIH3T3, neither the AH-D peptide nor the NH-D peptide has cytotoxicity.

FIG. 1C to 1E show the real-time interaction between surface-adsorbed dipalmitoylphosphatidylcholine (DOPC) liposomes and the AH-D or NH-D peptide on a titanium oxide-coated sensor chip monitored by QCM-D measurement. To this end, 0.1 mg/ml of the DOPC liposome was added at t=5 min to form a single layer of the surface-adsorbed liposome, and a 16 μM peptide was then added at t=50 min. The corresponding resonance frequency (FIG. 1B) and energy dissipation (FIG. 1C) were reported as a function of time. From the data of FIGS. 1C and 1D, a time-dependent plot of energy dissipation as a function of a resonance frequency shift is shown in FIG. 1E. Measurement was carried out in an aqueous solution containing 10 mM Tris and 150 mM NaCl at pH 7.4. The QCM-D kinetic profile is representative of four independent experiments.

1-2. Characterization of Melanoma Cell-Derived T-EXO

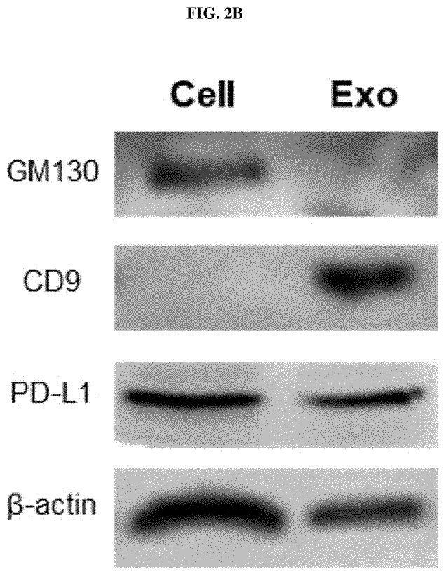

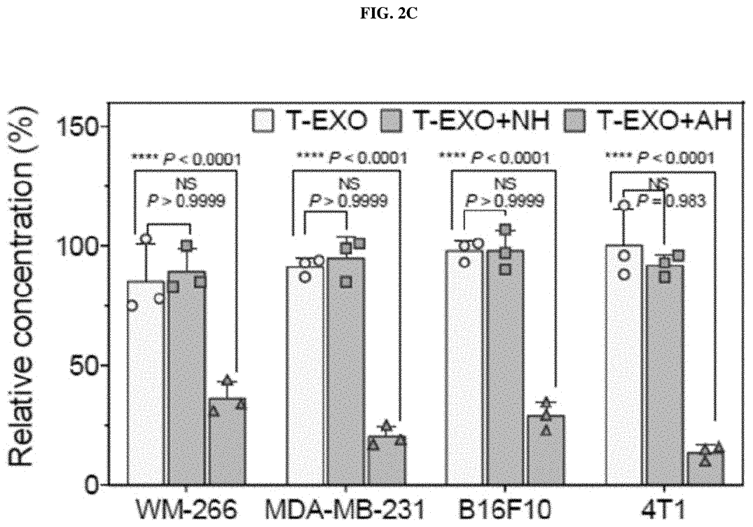

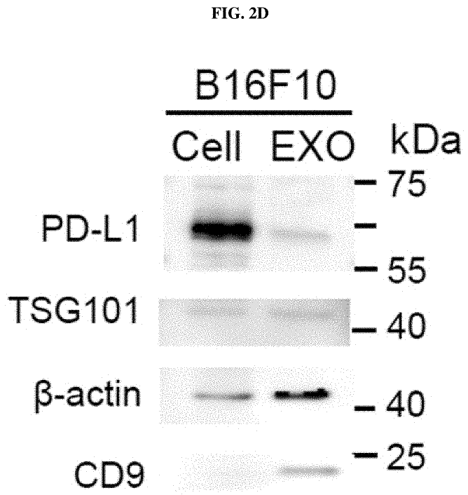

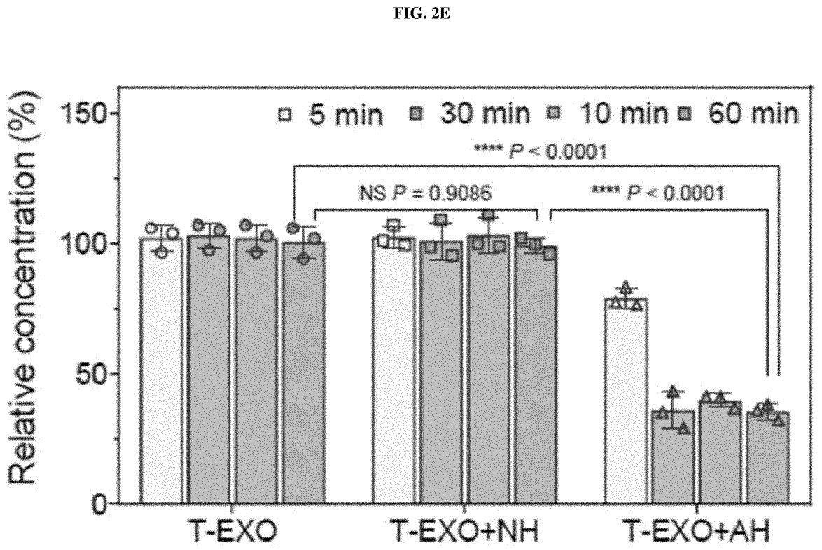

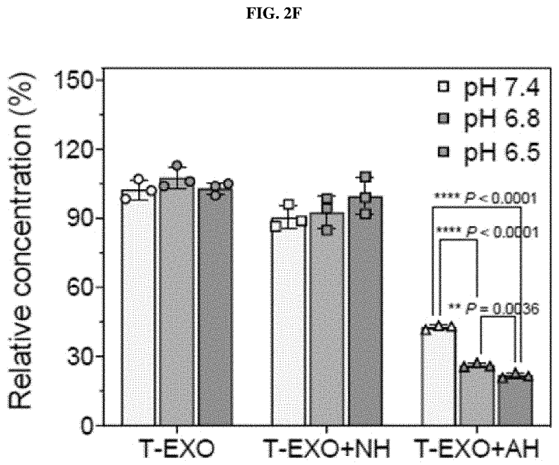

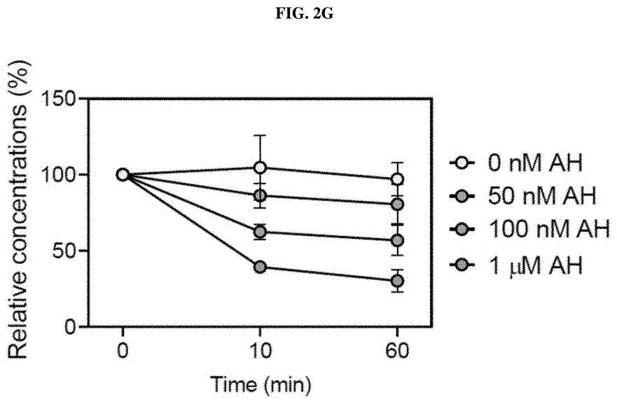

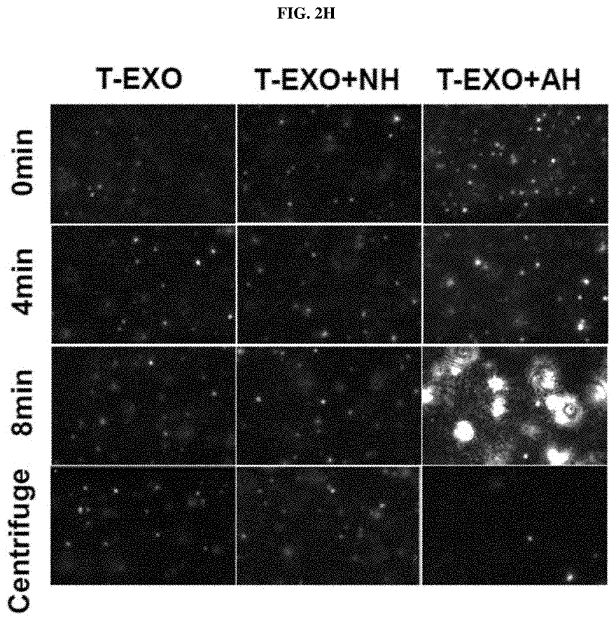

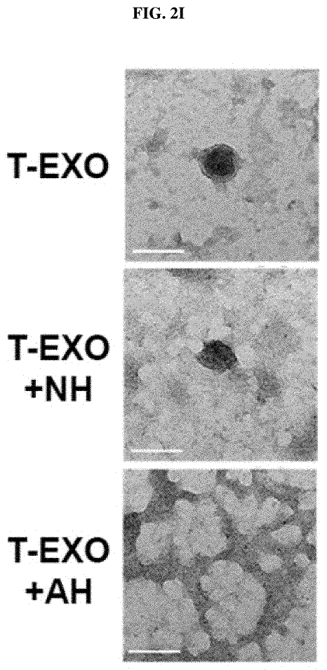

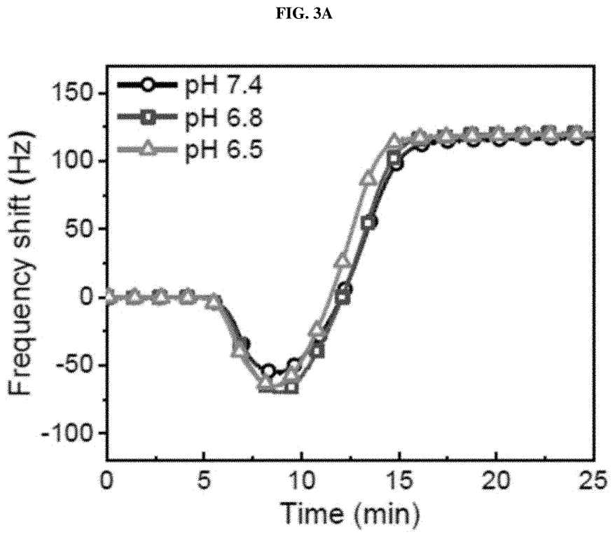

Through NTA analysis, as a result of confirming the size distribution of mouse melanoma B16F10 cell-derived T-EXO (the left panel of FIG. 2A) and the size distribution of T-EXO derived from various cells (WM-266 cells, MDA-MB-231 cells, and 4T1 cells) (the right panel of FIG. 2A), it was confirmed that they have a diameter of 50 to 200 nm.

In addition, to confirm the expression of a B16F10-derived T-EXO exosome biomarker and PD-L1, Western blotting was carried out. As a result, as shown in FIG. 2B, when comparing protein expression in a cell lysate and an exosome lysate, it was found that GM130, which is a cell membrane skeleton protein marker, appeared only in a cell lysate, whereas CD9 expressed on the membrane surface in an exosome formation process is expressed only in T-EXO. In addition, it was confirmed that the PD-L1 expressed on the melanoma cell surface is expressed not only in the cell lysate but also T-EXO.

1-3. Confirmation of Peptide Effect by NTA Analysis

Nanoparticle tracking analysis (NTA) was used to test the effect of AH-D and NH-D peptides on T-EXO with a diameter of 50 to 200 nm in various human and murine cancer cell lines.

According to T-EXO treatment with 1 μM of the AH-D or NH-D peptide for 10 minutes, followed by NTA, as shown in FIG. 2C, the AH-D peptide caused the disruption of T-EXO in all cases as indicated by a 65 to 85% decrease in the T-EXO number after 10-minute treatment, but the NH-D peptide did not show a significant effect.

Additional NTA experiments focused on murine melanoma B16F10 cell-derived T-EXO, and as shown in FIG. 2D, it was confirmed by Western blotting that a large amount of PD-L1^{EXO} was expressed on the cell surface of B16F10 cells.

In addition, as a result of NTA for T-EXO according to various times (FIG. 2E), pH conditions (FIG. 2F), or AH-D peptide conditions (FIG. 2G), as shown in FIG. 2E, the AH-D peptide rapidly disrupts T-EXO within 5 to 10 minutes, as shown in FIG. 2F, it was observed that T-EXO disruption was enhanced under acidic pH conditions (pH 6.5 and 6.8) related to an extracellular tumor microenvironment (TME), compared with the normal physiological condition (pH 7.4), and as shown in FIG. 2G, as the concentration of the AH-D peptide increases, a higher T-EXO disruption effect was exhibited. In addition, as shown in FIG. 2H, it was confirmed that T-EXO was removed by the AH-D peptide treatment in images captured by NTA.

As shown in FIG. 2I, transmission electron microscopy (TEM) images also provided the evidence that the AH-D peptide causes T-EXO disruption (scale bar=200 nm).

1-4. Confirmation of Result for Crystal Oscillator Microscale (QCM-D)

For further characterization of pH-enhanced membrane disruption, a QCM-D experiment was carried out to track the real-time interaction between the AH-D peptide and the surface-adsorbed liposome mimicking an exosome membrane under various pH conditions.

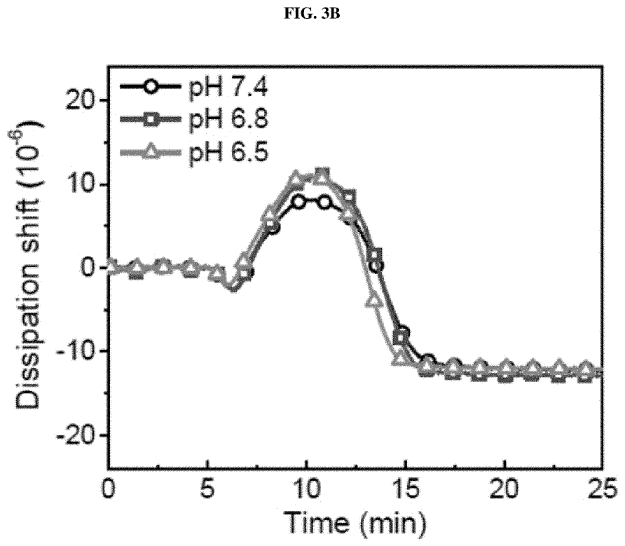

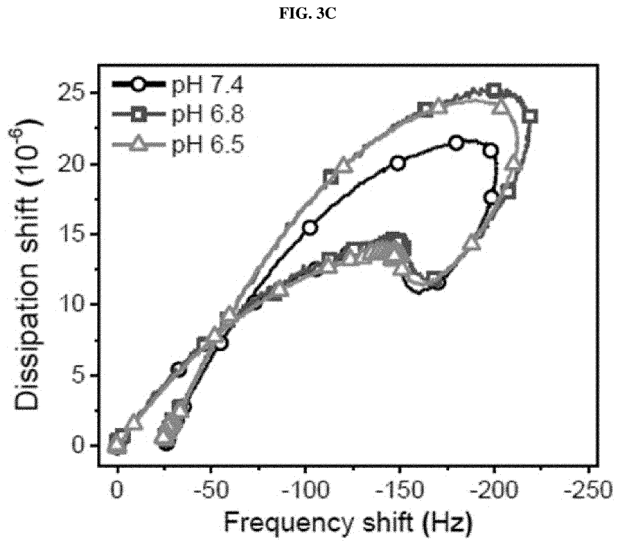

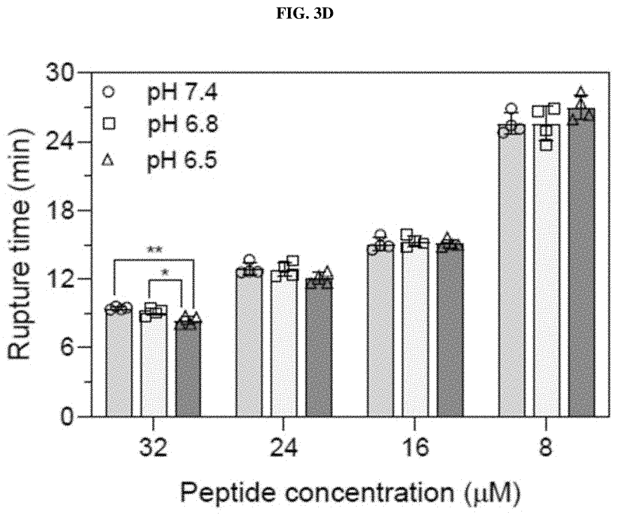

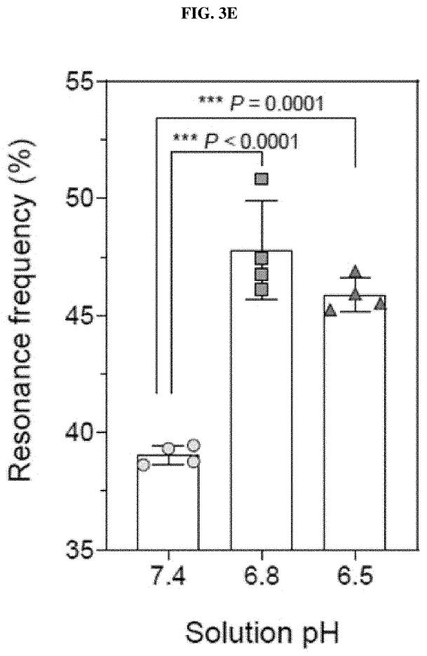

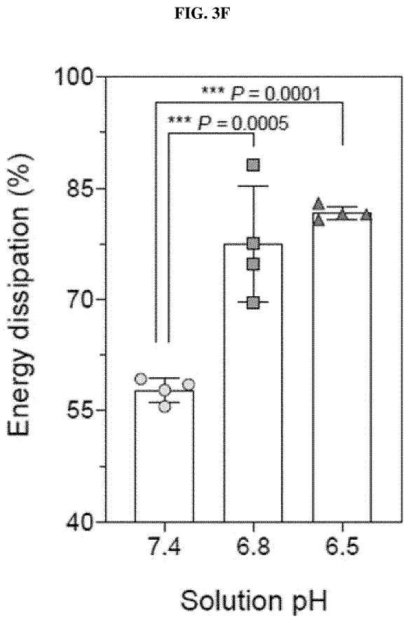

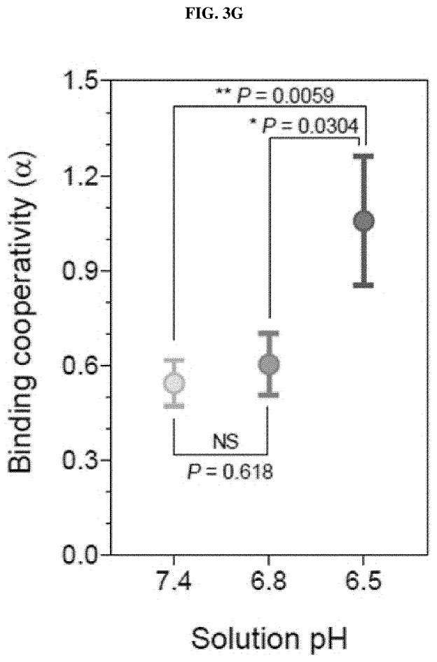

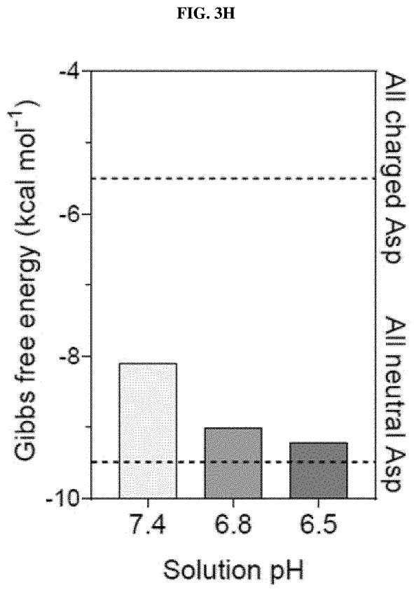

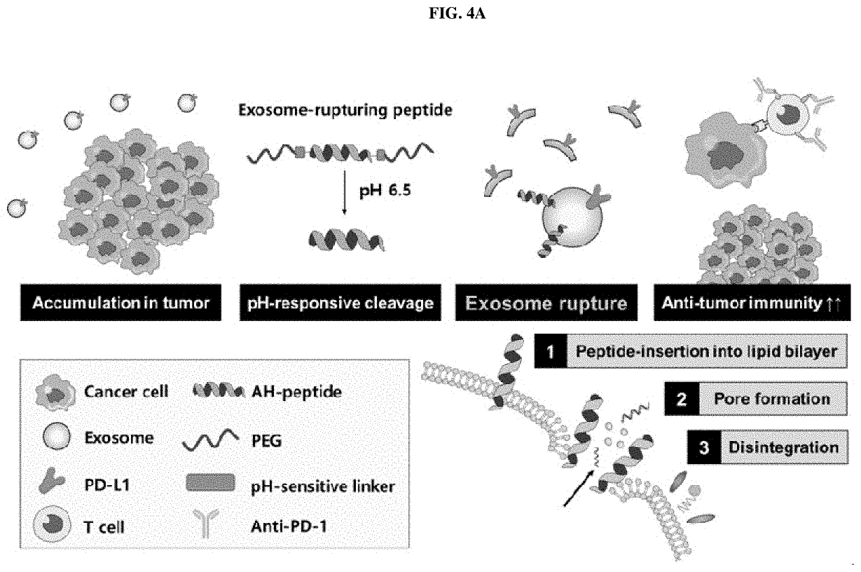

FIGS. 3A and 3B show QCM-D resonance frequency (FIG. 3A) and energy dissipation (FIG. 3B) shifts normalized as a function of time with respect to AH-D peptide-induced rupture of the surface-adsorbed liposome under various pH conditions, and from data of FIGS. 3A and 3B, a time-dependent plot of energy dissipation as a function of a resonance frequency shift is shown in FIG. 3C.

In FIG. 3D, an AH-D peptide-induced liposome rupture time according to a peptide concentration under various pH conditions was confirmed, and as the peptide concentration increased, the liposome rupture time decreased (n=4).

In addition, FIGS. 3E and 3F show that, when the AH-D peptide was added, the QCM-D resonance frequency (FIG. 3E) and the energy dissipation signal (FIG. 3F), corresponding to the mass and viscoelastic property of a liposome adlayer, respectively, undergo great changes exhibiting peptide-mediated disruption and ultimate rupture in all cases. Rather than at pH 7.4, at pH 6.5 and 6.8, more distinct changes were shown, and a wider range of membrane disruption was shown under an acidic condition.

In FIG. 3G, a phenomenological model was applied to analyze peptide-concentration-liposome rupture kinetics under various pH conditions, and it was confirmed that AH-D peptide binding cooperativity was greater at pH 6.5, which is associated with more preferable membrane disruption.

In FIG. 3H, as a result of estimating the predicted change in Gibbs free energy of membrane partitioning with respect to the AH-D peptide under various pH conditions based on the Wimley-White interfacial hydrophobicity scale, it was predicted that the AH-D peptide has three Asp residues sensitive to pH, generally has a pKa value of approximately 7 to 8 at an embedded interface, and the estimation shows the membrane partitioning at pH 6.5 and 6.8 being more favorable than that at pH 7.4 by approximately 1 kcal/mol. This thermodynamic difference arises from the pH-dependent change in the degree of ionization of the Asp residue, resulting in more Asp residues becoming neutral at a lower pH.

Collectively, these results supported that the AH-D peptide can disrupt T-EXO and exhibit pH-enhanced membrane disruption under an acidic condition associated with TME.

### Experimental Example 2. Characterization of PEG-Linker and PEG-Linker-Modified Peptide

2-1. ¹H-NMR Analysis of PEG-Linker

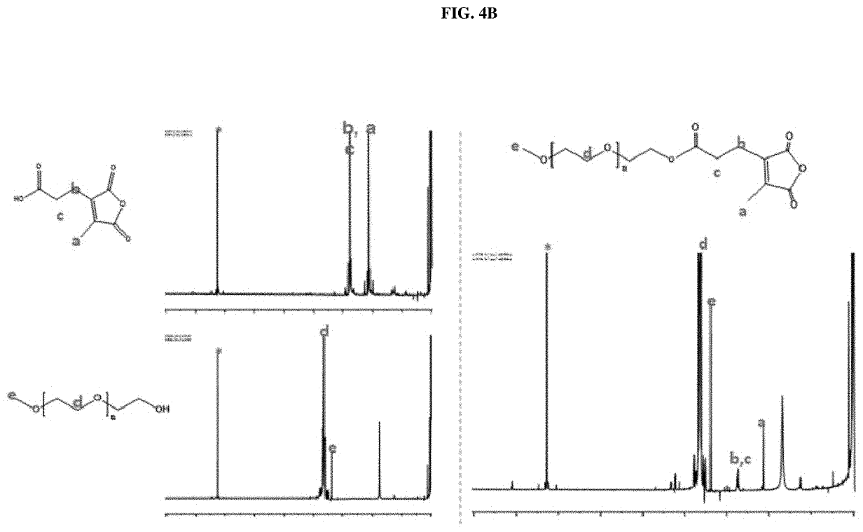

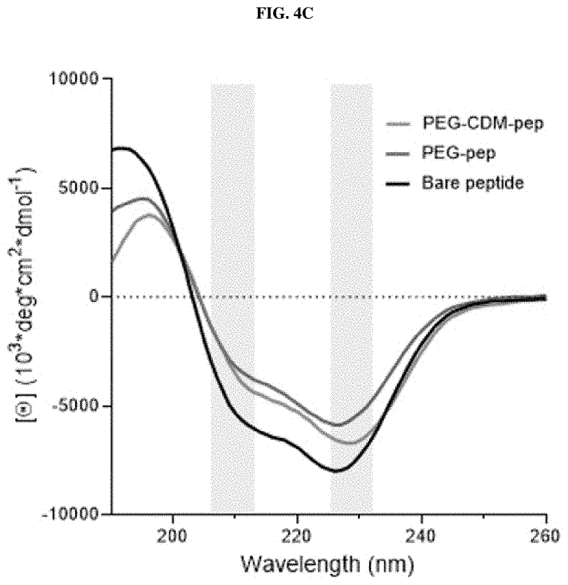

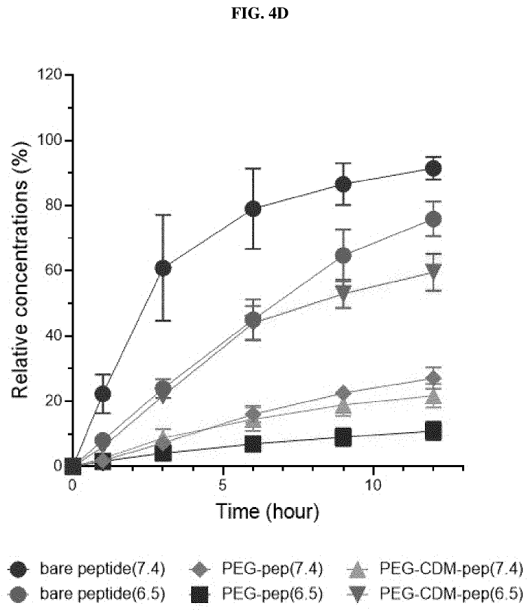

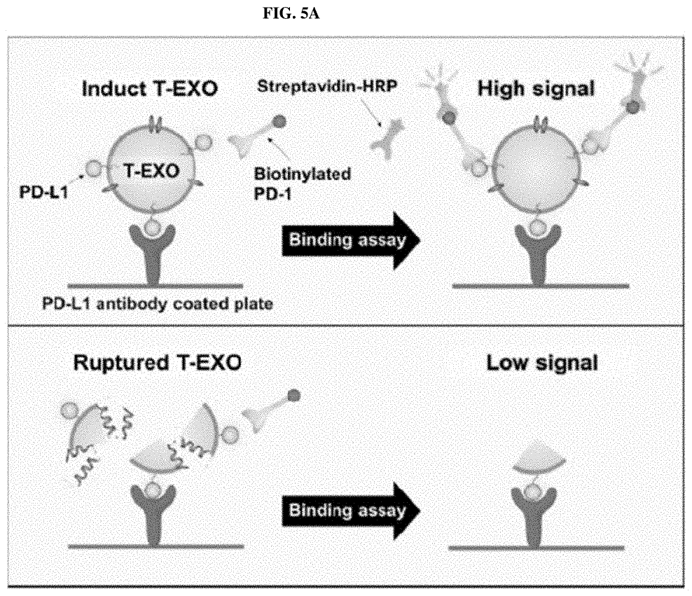

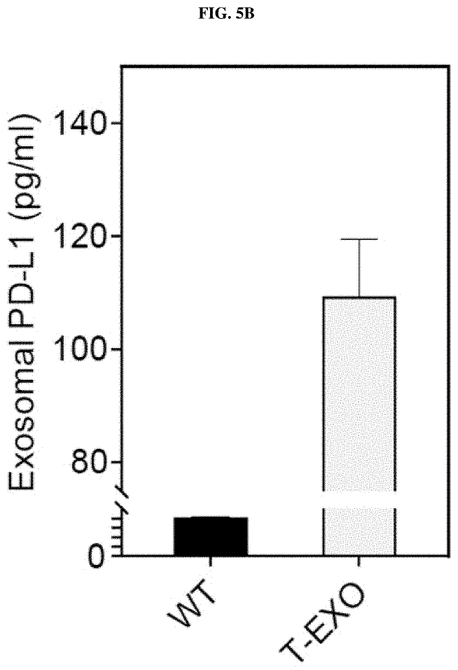

PEG-linker synthesis was confirmed by ¹H-NMR analysis: —C═C—CH₃ (δ=2.13 ppm), —CO—CH₂—CH₂— (δ=2.76 ppm), —CO—CH₂—CH₂— (δ=2.73 ppm), —CH₂—CH₂—O— (δ=3.64 ppm), —CH₃—O— (δ=3.38 ppm). As a result, as shown in FIG. 4B, by the ratio of the —C═C—CH₃ (δ=2.13 ppm) peak corresponding to the linker with respect to the width of the —CH₃—O— (δ=3.38 ppm) peak corresponding to the unique peak of PEG, it can be confirmed that the linker was bound to 95% or more of the PEG.

2-2. Circular Dichroism Spectroscopy for PEG-Linker-Modified Peptide

After modification of the AH peptide, circular dichroism spectroscopy was measured to confirm the maintenance of an α-helical secondary structure. Before modification, the spectrum of each of the AH peptide (Bare peptide), PEG-modified peptide (PEG-pep), and PEG-linker-modified peptide (PEG-CDM-pep) was observed, confirming that, as shown in FIG. 4C, all three showed the typical spectrum of the a-helical structure. As a result, it was confirmed that the modified peptide is also a structure capable of disrupting the phospholipid membrane of an exosome.

2-3. Confirmation of pH-Dependent Release Behavior of PEG-Linker-Modified Peptide

To confirm the pH sensitivity of PEG-linker-modified peptide, release behavior analysis was carried out using a membrane having a cutoff value of 7 kDa. To detect the released AH peptide, a cysteine moiety of the peptide was labeled with a phosphor, and PEG modification was attempted. Each peptide was dispersed in a phosphate buffered solution of a specific pH (pH 7.4 or 6.5) and contained in the membrane, and the amount of the peptide released over time was measured using an ultraviolet-visible spectrometer. As a result, as shown in FIG. 4D, at pH 7.4, approximately 21% of the peptide was released, but at pH 6.5, approximately 60% of the peptide was released for 12 hours. This is because, as the linker is disrupted by PEG-linker-modified peptide responding to a weak acidic environment, the AH peptide having a molecular weight of 3.2 kDa passed through the membrane having a cutoff value of 7 kDa. Therefore, it can be seen that the synthesized PEG-linker-modified peptide responded to the tumor microenvironment.

The schematic diagram of the action of PEG-linker-modified peptide is shown in FIG. 4A.

### Experimental Example 3. Inhibition of T-EXO-Mediated T Cell Functional Impairment

Since the membrane environment of PD-L1^{EXO} affects structural properties, the effect of the AH-D peptide on the PD-1 binding ability of T-EXO was investigated by ELISA.

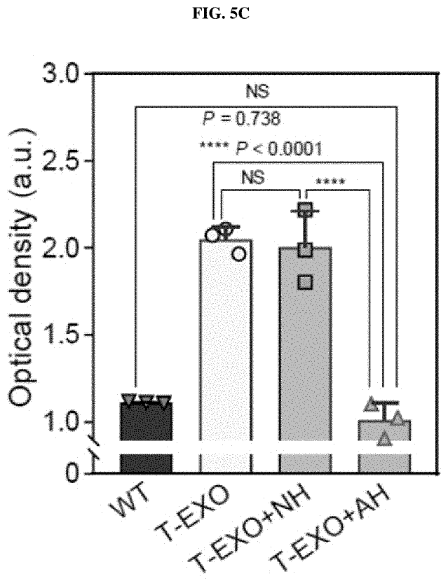

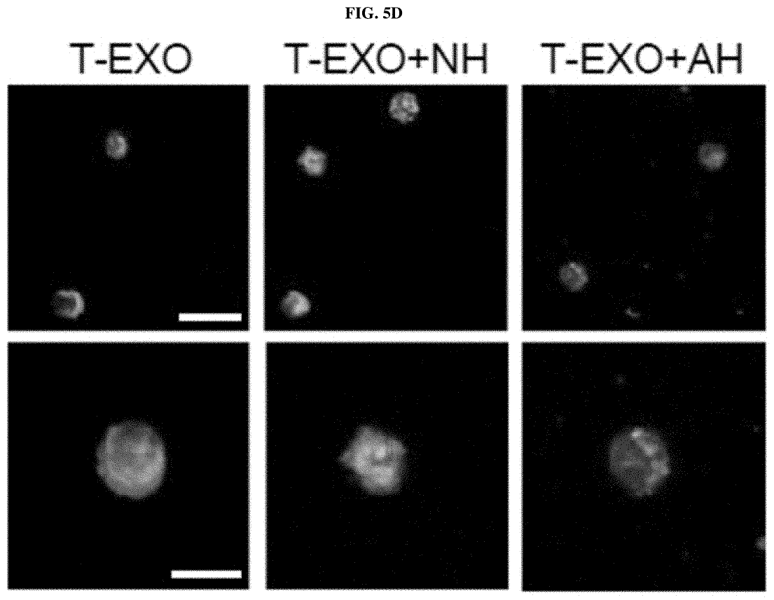

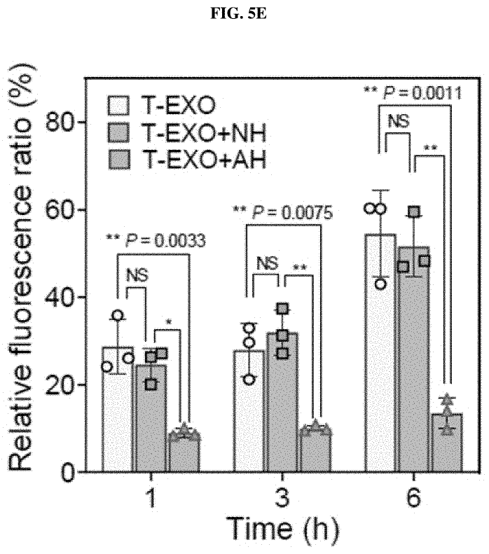

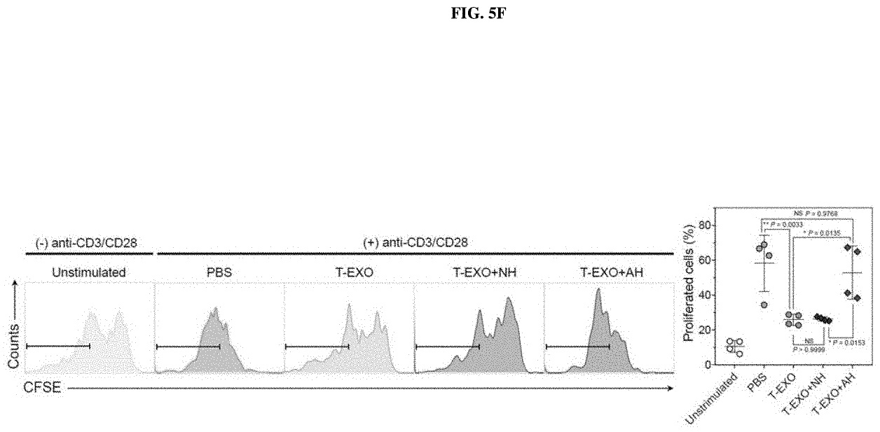

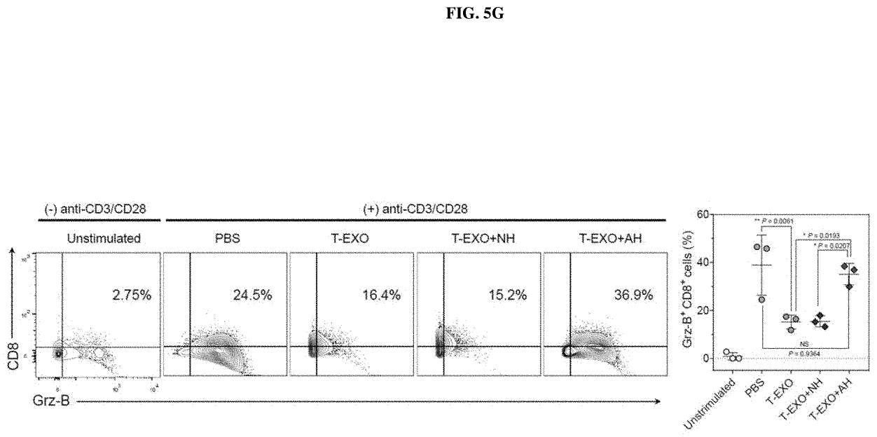

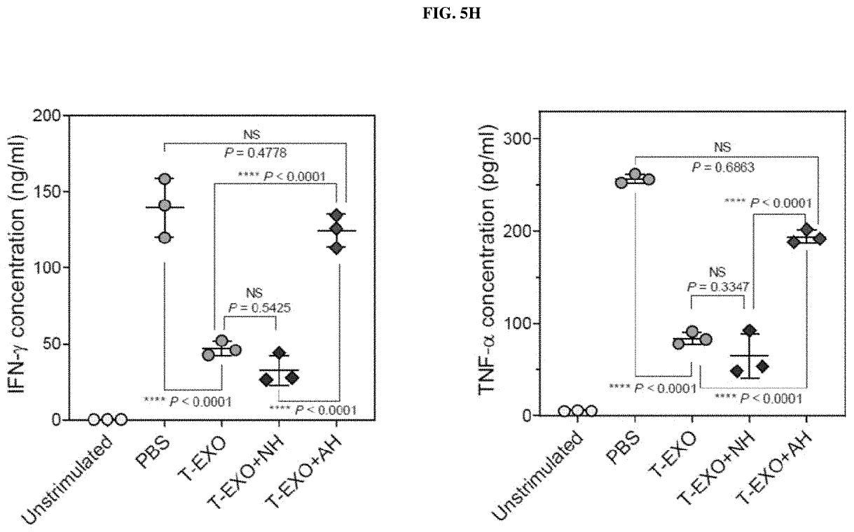

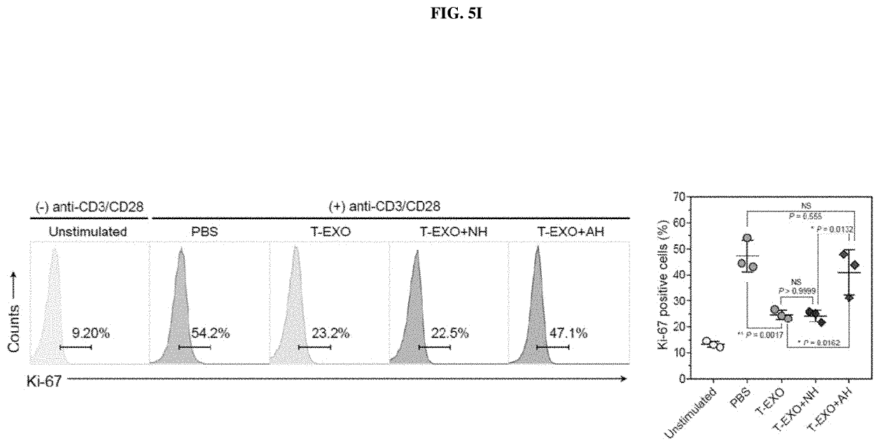

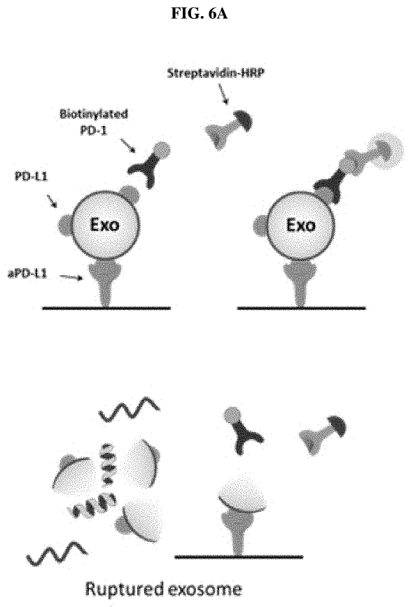

FIG. 5A shows the schematic diagram of the PD-1 binding of T-EXO and its action.

In FIG. 5B, it was confirmed that the PD-L1 content was higher in T-EXO isolated from the plasma of a B16F10 tumor-bearing mouse than in the wild-type, and in FIG. 5C showing a result of the AH-D peptide treatment on PD-L1-containing T-EXO isolated from the plasma of the B16F10 tumor-bearing mouse, it was confirmed that the binding ability of T-EXO by PD-L1 was significantly reduced to the level of EXO isolated from the plasma of a wild-type mouse. In contrast, NH-D peptide treatment did not affect the binding ability of T-EXO to PD-1.

In addition, to evaluate the endocytic uptake of T-EXO by CD8⁺ T cells occurring by PD-1 cell receptor binding, confocal microscopy was performed. In FIG. 5D, as a result of the observation of confocal microscope images, it was confirmed that the T cell uptake of T-EXO treated with the AH-D peptide was significantly reduced compared to T-EXO not treated with a peptide and T-EXO treated with the NH-D peptide (scale bar=10 μm, blue: cell nucleus, red: Cy5.5-labeled T-EXO), and in FIG. 5E, it was confirmed that the difference in relative fluorescence intensity with the NH-D peptide-treated group increases over time.

These results are consistent with the loss of PD-1 binding ability by PD-L1^{EXO} upon AH-D peptide treatment.

In addition, to confirm whether the AH-D peptide can inhibit the T cell functional impairment induced by T-EXO, flow cytometry was carried out to measure CD8⁺ T cell proliferation. As a result, in FIG. 5F, it was confirmed that, while the T-EXO not treated with a peptide reduces the proliferation potential of T cells, T cells exposed to the T-EXO treated with the AH-D peptide have high proliferation potential similar to normally activated T cells.

FIG. 5G shows that the inhibition of the cytotoxic function of activated T cells by T-EXO is prevented by AH-D peptide treatment. Compared to the normally-activated T cells, the T cells exposed to the AH-D peptide-treated T-EXO showed a similar fraction of CD8⁺ T cells expressing granzyme B (GrzB) secreted in T cell activation, whereas the T cells exposed to untreated or NH-D peptide-treated T-EXO showed a significantly lower fraction of the cells.

Further, in FIGS. 5H and 5I, compared to T cells incubated with the untreated or NH-D peptide-treated T-EXO, it was confirmed that pro-inflammatory cytokines, that is, interferon-gamma (IFN-γ) and tumor necrosis factor-alpha (TNF-α) are produced (FIG. 5H) and Ki-67-expressing T cells are further increased by T cells incubated with the AH-D peptide-treated T-EXO (FIG. 5I).

These results supported the fact that the AH-D peptide inhibits T-EXO in vitro, resulting in loss of PD-L1^{EXO} binding ability to T cells, thereby preventing T cell depletion.

### Experimental Example 4. Inhibition of T-EXO-Mediated T Cell Functional Impairment of PEG-Linker-Modified Peptide

4-1. Confirmation of T-EXO Disruption Effect of PEG-Linker-Modified Peptide

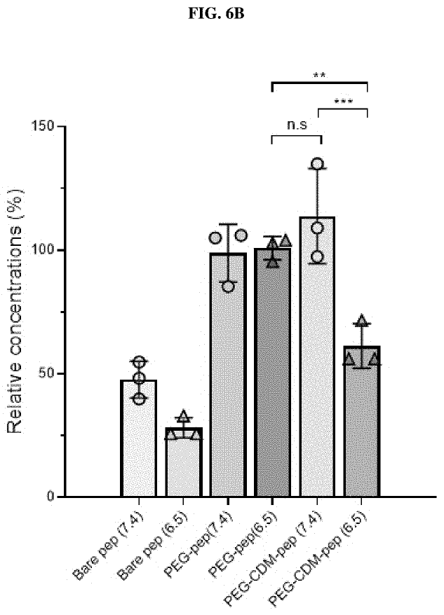

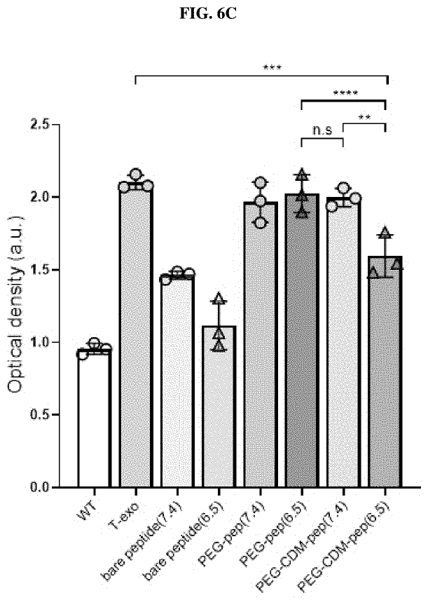

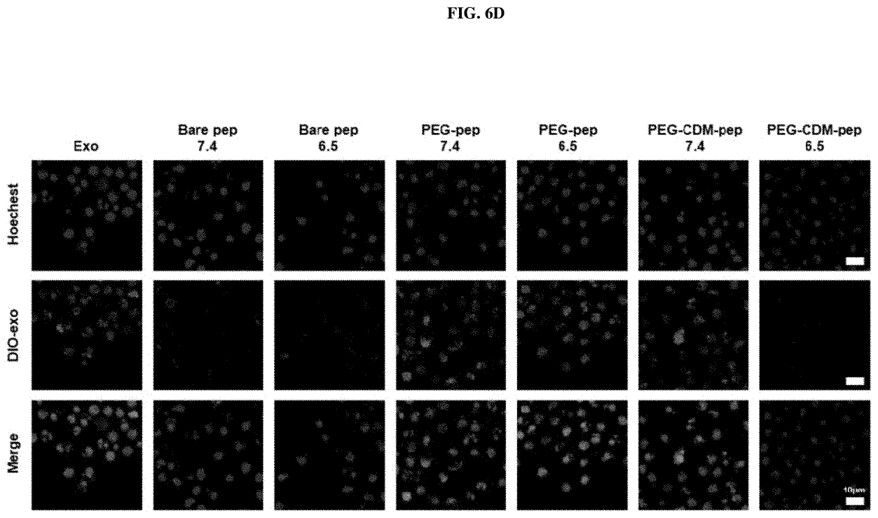

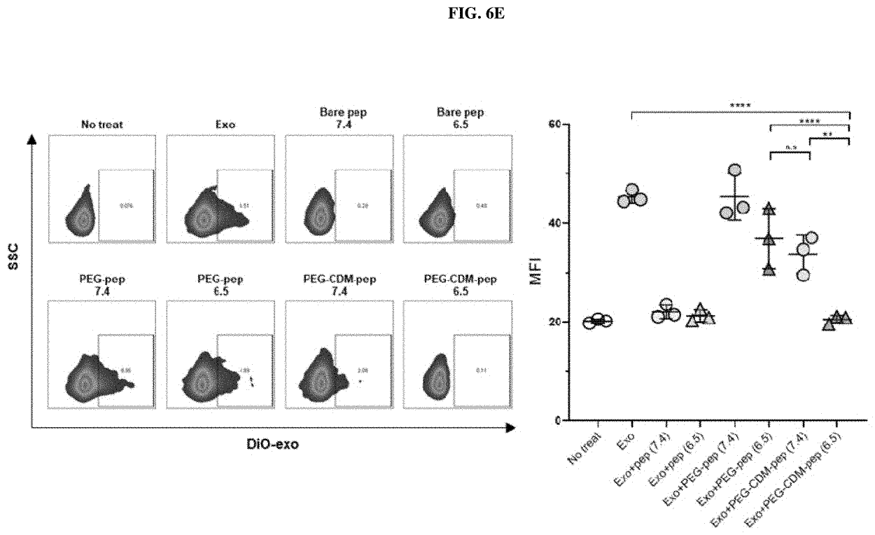

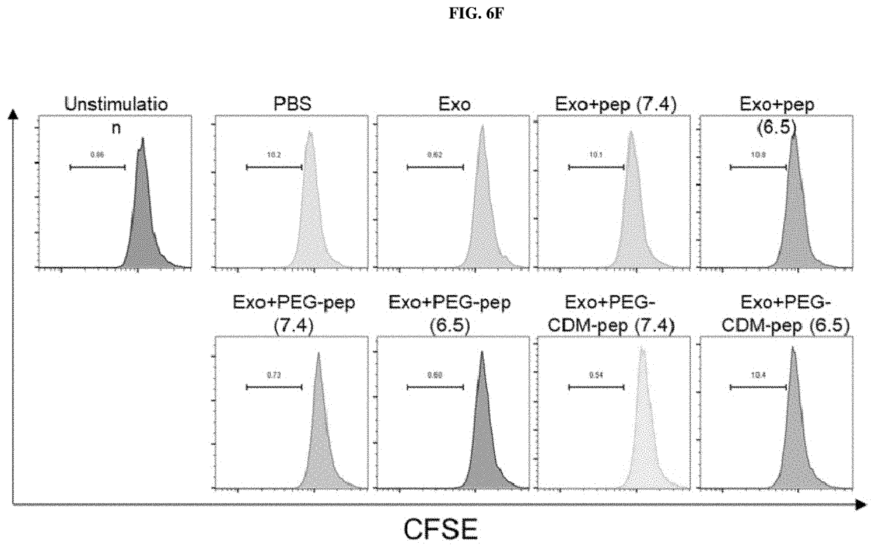

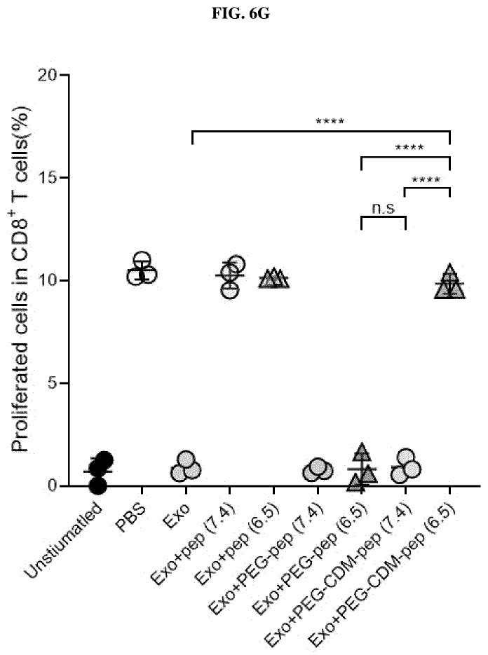

The schematic diagram of the process of PD-1/PD-L1 binding ability analysis to confirm the T-EXO disruption effect of the peptide is shown in FIG. 6A. As a result of evaluating the T-EXO disruption level using NTA, as shown in FIG. 6B, it can be seen that, unlike pH 7.4, at pH 6.5, as the AH peptide was released by the disruption of a linker, T-EXO was disrupted. In contrast, it can be confirmed that PEG-modified peptide did not exhibit a disruption effect by binding of the active moiety of the AH peptide to PEG.

4-2. Confirmation of PD-1/PD-L1 Binding Inhibitory Effect of PEG-Linker-Modified Peptide

ELISA was performed to investigate the change in PD-1/PD-L1 binding force by T-EXO membrane disruption.

As a result of measuring the degree of PD-L1 on an exosome surface by ELISA, as shown in FIG. 6C, it was confirmed that PEG-linker-modified peptide inhibited PD-1/PD-L1 binding only in an environment of pH 6.5. This is because, like the result shown in FIG. 6B, the AH peptide is released by disruption of the linker reacting only in a tumor microenvironment, that is, pH 6.5.

In addition, to evaluate the T cell uptake of T-EXO, confocal microscopy was used. As a result of observation of the microscope images, as shown in FIG. 6D, it was confirmed that not only the cellular uptake of the AH peptide-treated T-EXO was decreased, but also cellular uptake of PEG-linker-modified peptide was selectively reduced in an environment of pH 6.5 (Scale bar=10 μm, blue: cell nucleus, green: DiO phosphor-labeled T-EXO). This is because, unlike PEG-modified peptide, PEG-linker-modified peptide selectively released the AH peptide only in an environment of pH 6.5 to disrupt the T-EXO membrane, resulting in inhibiting PD mediated cellular uptake on the T cell membrane surface.

As a result of flow cytometry for the same sample group to quantify the experimental result shown in FIG. 6D, as shown in FIG. 6E, it was confirmed that the cellular uptake of the PEG-linker peptide is inhibited at a similar level to that of an unmodified AH peptide at pH 6.5.

4-5. Confirmation of Inhibition of T-EXO-Mediated T Cell Functional Impairment

To confirm whether PEG-linker-modified peptide can inhibit T cell functional impairment induced by T-EXO, a pre-treated peptide and T-EXO were used to estimate the effect on CD8⁺ T cell proliferation at pH 7.4 or 6.5. As a result, as shown in FIGS. 6F and 6G, it was shown that PEG-linker-modified peptide can disrupt T-EXO only in a tumor-specific environment, that is, pH 6.5, thereby degrading T cell functional impairment caused by T-EXO.

### Experimental Example 5. Cytotoxicity Test

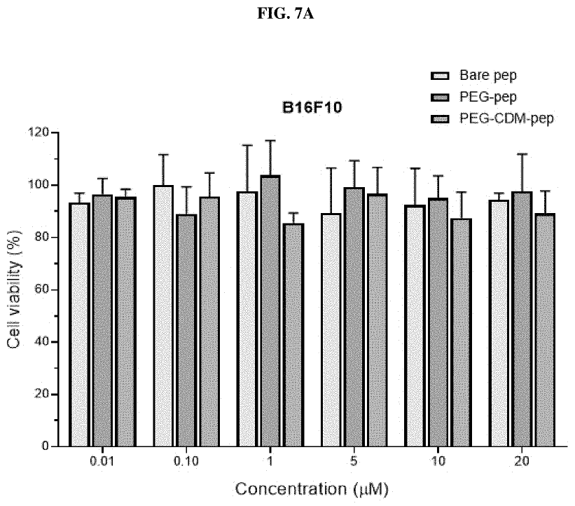

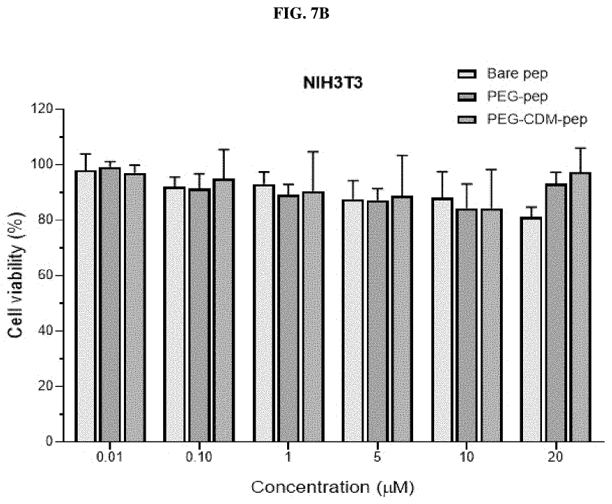

To confirm whether the α-helical structure of a peptide exhibits cytotoxicity due to the modification of a cell membrane structure, a cytotoxicity test for the AH peptide, PEG-modified peptide, and PEG-linker-modified peptide was carried out. The test was carried out for each of a mouse-derived melanoma cell line B16F10 and mouse-derived fibroblasts NIH3T3. Consequently, it can be seen that, as shown in FIGS. 7A and 7B, not only the AH peptide but also PEG-modified peptide and PEG-linker-modified peptide did not exhibit cytotoxicity.

### Experimental Example 6. Evaluation of Animal Blood T-EXO Level According to Concentration of PEG-Linker-Modified Peptide

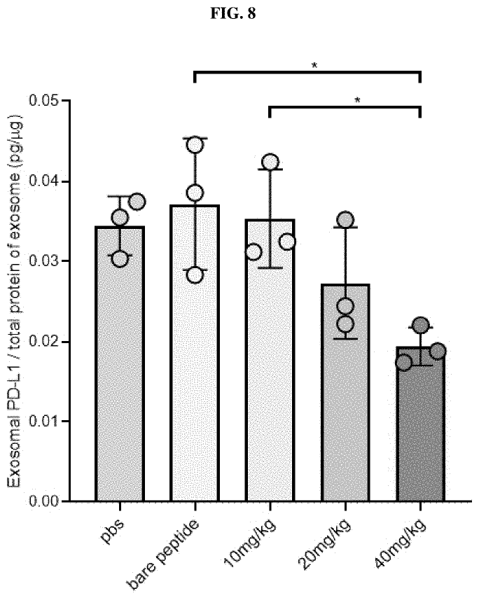

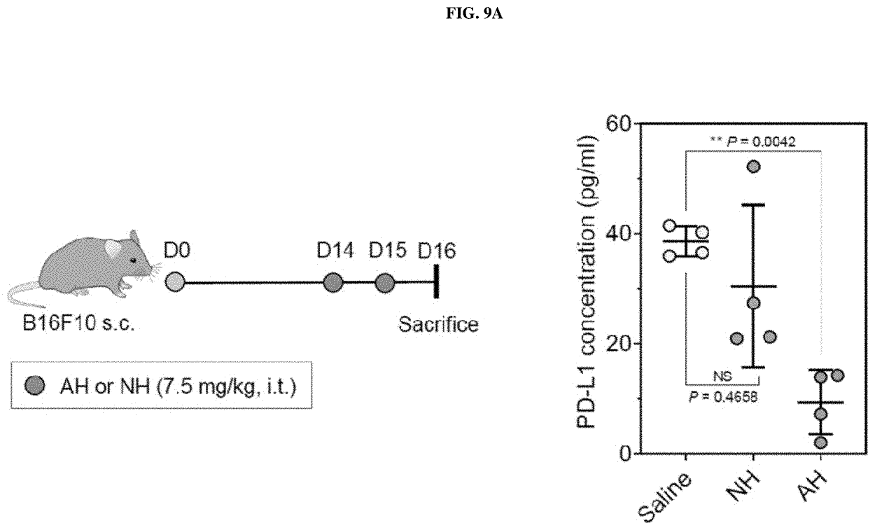

In a tumor animal model, the amount of blood T-EXO according to the intravenous injectable concentration of PEG-linker-modified peptide was assessed by ELISA. As a result of confirming the ratio of the T-EXO amount in tumor tissue by showing the ratio of a PD-L1 expression level with respect to a protein level of total exosomes isolated from tumor tissue, as shown in FIG. 8, it can be confirmed that, when PEG-linker-modified peptide (40 mg/kg) was intravenously injected, the ratio of the tumor tissue T-EXO was reduced by approximately 45%. Therefore, compared to the AH peptide, it can be seen that PEG-linker-modified peptide was improved in biostability by PEG modification, had tumor microenvironment sensitivity due to the linker, and disrupted T-EXO.

### Experimental Example 7. Improvement in Antitumor Effect by PD-1 Blocking

In the case of a PD-1 blocking therapy, since it is predicted that a high level of PD-L1^{EXO} before treatment will exhibit a low treatment response, whether early AH-D peptide treatment can improve the therapeutic result of an antibody-based PD-1 inhibitor (aPD-1) was investigated.

First, the effect of AH-D peptide therapy alone on the level of circulating PD-L1^{EXO} and an immunosuppressive TME phenotype formed by T-EXO in the B16F10 tumor-bearing mouse were assessed. Particularly, in FIG. 9A, it was confirmed that the AH-D peptide therapy alone reduces the level of PD-L1^{EXO} circulating in vivo by approximately 76%, compared to a physiological saline-treated group.

Referring to FIG. 9B, it was confirmed that the intratumoral population of immunosuppressive cells including CD4⁺ CD25⁺ regulatory T cells (Tregs) and CD11b⁺Gr-1⁺ bone marrow-derived suppressor cells (MDSCs) is also significantly reduced in an AH-D peptide-treated mouse or compared to a saline-treated or NH-D peptide-treated mouse. Transforming growth factor-β (TGF-β) is a major tumor-promoting cytokine that induces exhausted T cells and forms a fibrous TME, and the expression level thereof in a tumor of the AH-D peptide-treated mouse was reduced by approximately 40% compared to a tumor of the saline-treated mouse. This demonstrates that the AH-D peptide therapy alone can reduce circulating PD-L1^{EXO} and the level of immunosuppressive cells in tumors.

In addition, the antitumor efficacy of the AH-D peptide and aPD-1 antibody co-administration therapy was evaluated. Mice were inoculated with B16F10 melanoma cells, 12 days after inoculation, a treatment regimen began with saline, the NH-D peptide (NH), the AH-D peptide (AH), aPD-1, the NH-D peptide and an aPD-1 antibody (NH+aPD-1), or the AH-D peptide and the aPD-1 antibody (AH+aPD-1), and such a protocol is schematically shown in FIG. 9C. In the treatment group, peptide treatment began two days before antibody treatment to lower the level of circulating PD-L1^{EXO} and attenuate tumors.

As a result, FIGS. 9D and 9E show that, compared to the saline-treated control, the AH and NH groups had a little effect on the tumor growth profile, and there is no significant difference in tumor volume (FIG. 9D) and weight (FIG. 9E) in the NH+aPD-1 group, compared to the aPD-1 group. In contrast, the AH+aPD-1 group showed significant decreases in tumor volume and weight of more than 65% and 80%, respectively, compared to the aPD-1 group.

From these results, it was seen that the AH+aPD-1 co-administration therapy have superior antitumor efficacy compared to aPD-1 therapy alone.

In addition, in FIGS. 9F and 9G, H&E staining (top) and MT staining (bottom) results (scale bar=200 μm) in cancer tissues and a H&E staining result in major organs were confirmed (scale bar=100 μm), and FIG. 9H shows a change in weight of a mouse, confirming that there were no significant changes in weight and major organ histology and all treatment regimens were well tolerated.

T-EXO is involved in various immune checkpoint blockade (ICB)-resistant mechanisms including transformation of fibroblasts into cancer-associated fibroblasts (CAFs) stimulating angiogenesis that can prime a fibrotic TME and prevent T cell invasion into tumors. Accordingly, in FIG. 9I, immunofluorescent staining of CAF markers, including α-smooth muscle actin (α-SMA) and fibronectin, and the angiogenesis marker CD31, was performed on tumor tissue along with histological staining, and thus it was confirmed that AH-D peptide treatment effectively reconstituted a local TME and prevented fibrosis and angiogenesis (scale bar=50 μm).

Collectively, these results supported that the AH-D peptide treatment enhanced the antitumor response of an aPD-1 antibody and remodeled the TME into a more immunological phenotype.

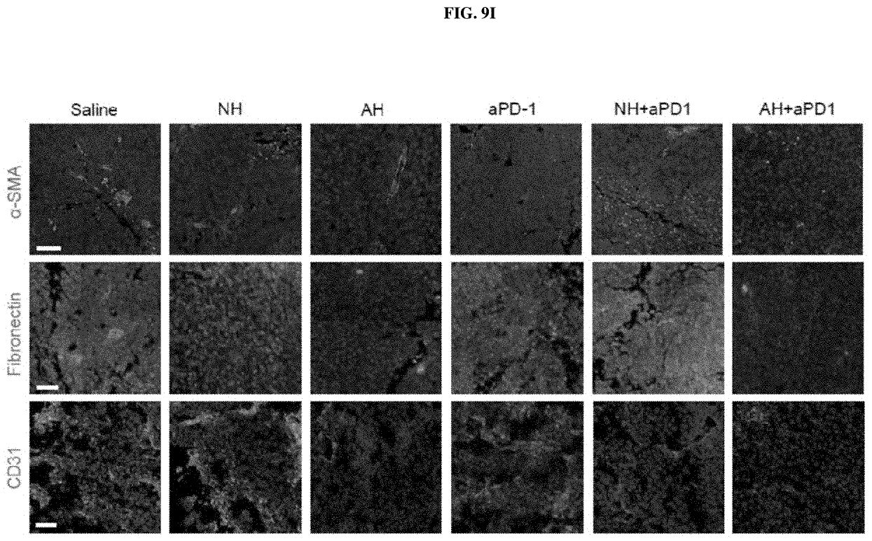

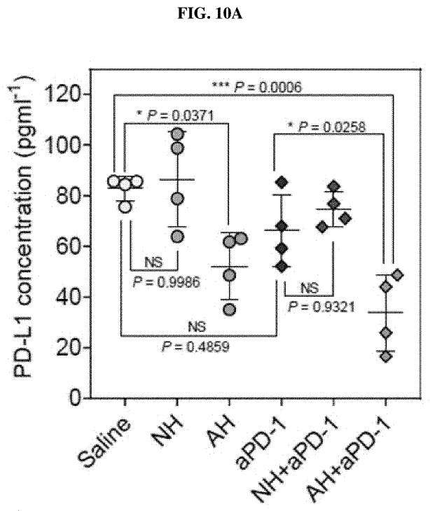

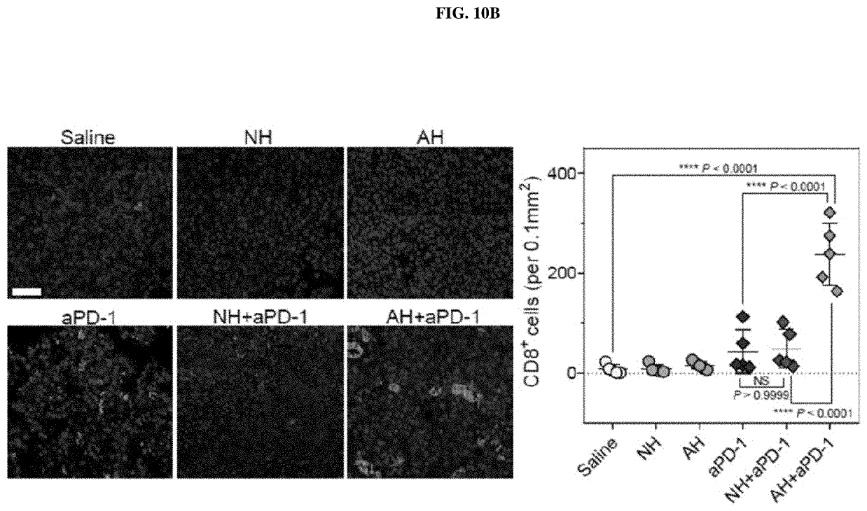

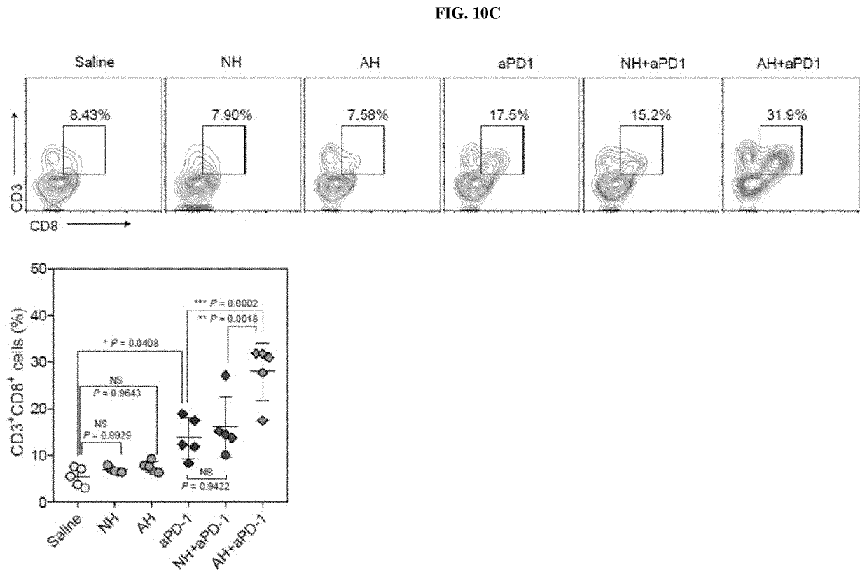

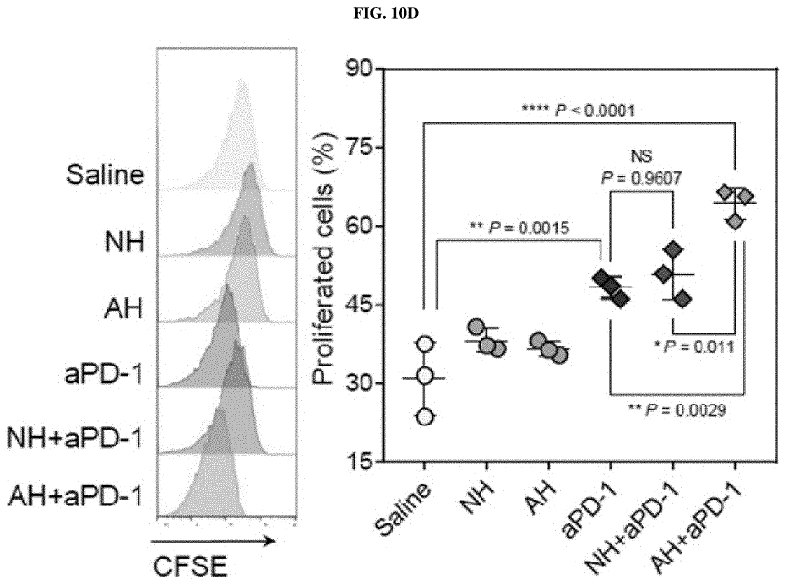

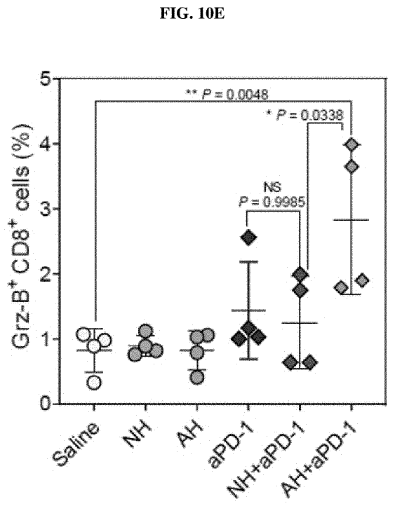

Through further analysis, in FIG. 10A, it was confirmed that, compared to the saline-treated group, the AH group decreased a circulating PD-L1^{EXO} level, and compared to the aPD-1 group, the AH+aPD-1 group decreased a PD-L1^{EXO} level.

Referring to FIG. 10B, the immunofluorescence staining result proved that the AH+aPD-1 group significantly increased the homing of CD8⁺ cells to a tumor site, compared to all other groups (scale bar=50 μm). This result was consistent with the remodeled TME that allowed T cell entry due to T-EXO depletion.

Referring to FIG. 10C, it was confirmed that the frequency of CD3⁺ CD8⁺ T cells in the tumor tissue of the AH-aPD-1 group was more than twice that of the aPD-1 group. Further, referring to FIGS. 10D and 10E, the AH+aPD-1 group showed a high level of T cell proliferation potential (FIG. 10D) and a high frequency of (GrzB)-expressing CD8⁺ T cells (FIG. 10E), indicating that antitumor immunity was improved compared to the aPD-1 group. These results supported that the AH-D peptide amplifies the antitumor reaction of PD-1 blockade.

### Experimental Example 8. Inhibition of Pre-Metastatic Niche Formation

In addition to promoting tumor progression, T-EXO plays an important role in metastasis by constructing a pre-metastatic niche that promotes the efflux and colonization of cancer cells in secondary organ sites.

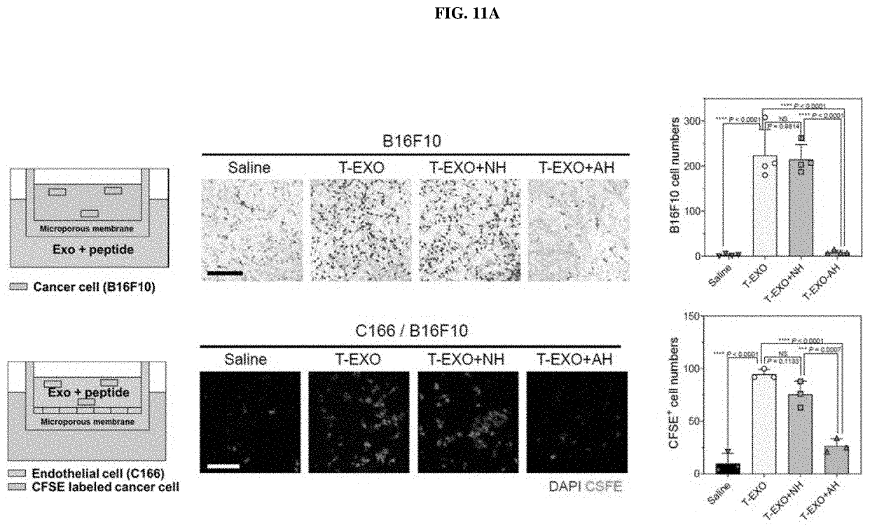

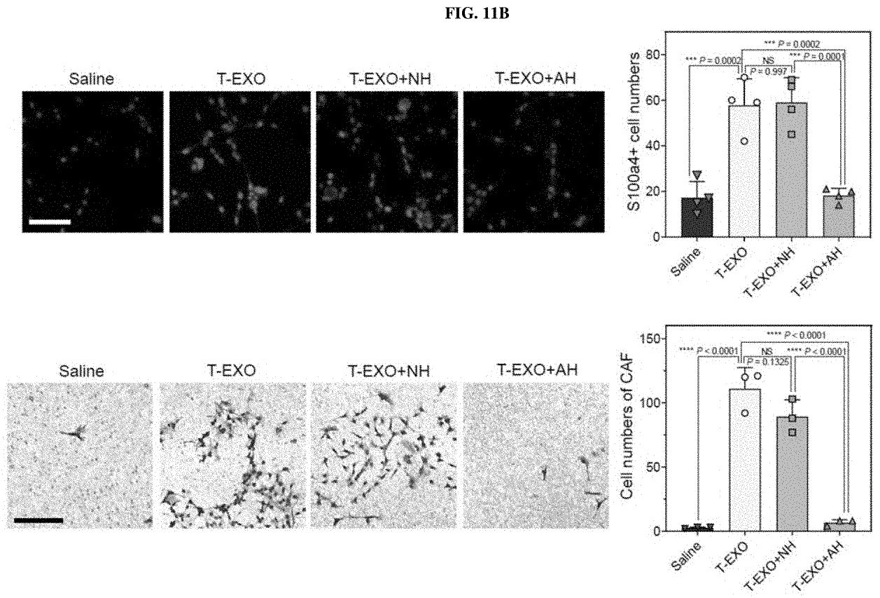

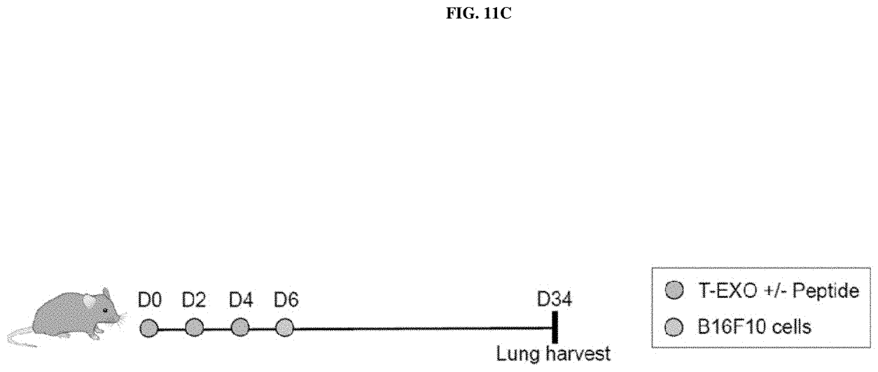

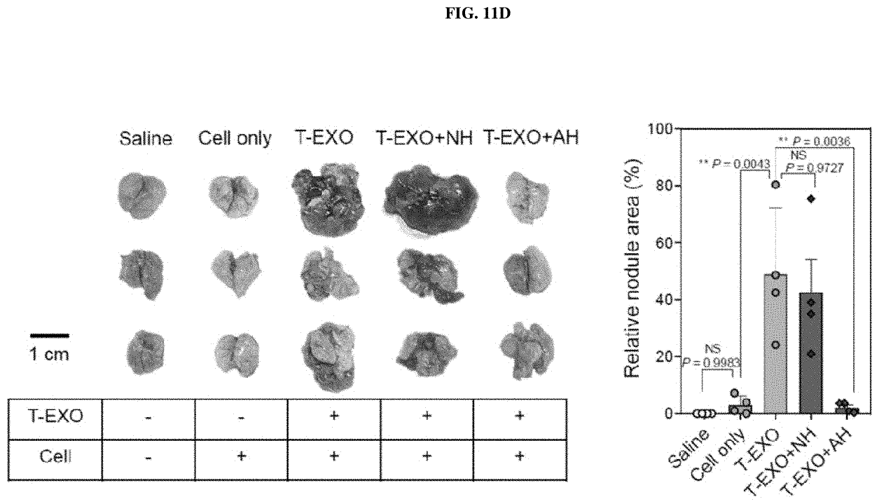

Referring to FIG. 11A, as a result of in vitro migration analysis to evaluate the metastasis potential of cancer cells in the presence of T-EXO, as expected, it was confirmed that the number of migrated B16F10 cells was particularly high upon the addition of T-EXO known to increase endothelial permeability (top of FIG. 11A). When T-EXO was treated with the AH-D peptide, compared to that observed in the NH-D peptide-treated group, the transendothelial migration of B16F10 cells was significantly inhibited by 35% (bottom of FIG. 11A).

In addition, referring to FIG. 11B, the metastasis promoting role of T-EXO was further investigated by measuring in vitro activation of CAF cells involved in cancer metastasis. As a result, when the expression of S100A4, which is a major regulator of lung-directed metastasis, was observed by a confocal microscope, it was highly upregulated in the presence of T-EXO alone and significantly reduced in the presence of T-EXO treated with the AH-D peptide (top of FIG. 11B, blue: cell nucleus, red: S100A4, scale bar=100 μm). In addition, when T-EXO was treated with the AH-D peptide, it was confirmed by Giemsa staining that the number of pro-migratory CAF cells significantly decreases, this supported the decreased activation of CAF cells (bottom of FIG. 11B, scale bar=1000 μm).

Based on these results, the in vivo anti-metastatic effect of the AH-D peptide was further evaluated in vivo. To investigate the role of circulating T-EXO that forms a pre-metastatic niche, T-EXO was intravenously administered into mice every two days in the presence or absence of the AH-D or NH-D peptide (T-EXO+AH or T-EXO+NH), and tumor-induced B16F10 cancer cells were injected. The aforementioned experimental protocol is shown in FIG. 11C.

Referring to FIG. 11D, as a result of examining the lungs extracted 4 weeks after administration, it was confirmed that, when T-EXO and cancer cells are administered, compared to when only cancer cells are administered, tumor growth was promoted, and this result supported that T-EXO accelerates tumor metastasis by priming the microenvironment of a distant organ to make it advantageous to propagate and colonize cancer cells. In contrast, AH-D peptide treatment effectively inhibited the metastasis promotion of T-EXO, and the T-EXO+AH group showed low lung metastasis, similar to the cell-only group, but the T-EXO-NH group showed high metastasis.

These results were also supported by the histological analysis of a metastatic lesion shown in FIG. 11E (top, scale bar=500 μm) and immunofluorescence staining of a lung tissue section (bottom, scale bar=100 μm). The histological analysis is the H&E staining result for a lung tissue section, and the black arrow indicates a metastatic nodule. In relation to lung metastasis, it has been reported that activated fibroblasts expressing S100A4 and α-SMA are required for metastatic colony formation, and fibronectin is upregulated during pre-metastatic niche formation. This tendency was consistent with the experimental results of the present invention as the expression levels of S100A4, α-SMA and fibronectin significantly increased in the T-EXO and T-EXO+NH groups. On the other hand, the expression levels of the markers in the T-EXO+AH group were similar to that of the cell-only group, and a similar tendency was observed in liver micrometastasis induced by T-EXO.

In addition, from the above results, it was seen that the AH-D peptide treatment effectively inhibited the metastasis promoting function of T-EXO.

It should be understood by those of ordinary skill in the art that the above description of the present invention is exemplary, and the example embodiments disclosed herein can be easily modified into other specific forms without changing the technical spirit or essential features of the present invention. Therefore, it should be interpreted that the example embodiments described above are exemplary in all aspects, and are not limitative.

1. A peptide for cancer immunotherapy, comprising:
an amino acid sequence of SEQ ID NO: 1.

2. The peptide of claim 1, wherein the peptide has an α-helical structure.

3. The peptide of claim 1, wherein the peptide disrupts tumor-derived vesicles.

4. The peptide of claim 1, wherein the peptide inhibits T cell functional impairment.

5. The peptide of claim 1, wherein the peptide controls a tumor microenvironment.

6. The peptide of claim 1, wherein the peptide inhibits the angiogenesis or fibrosis of tumors.

7. The peptide of claim 1, wherein the peptide comprises a peptide modified with polyethylene glycol (PEG).

8. The peptide of claim 7, wherein the polyethylene glycol is bound to the peptide via a linker.

9. The peptide of claim 8, wherein the linker is sensitive to the tumor microenvironment.

10. The peptide of claim 9, wherein the linker is a cleavable linker cleaved in response to the tumor microenvironment.

11. A method of cancer immunotherapy, comprising:
administering the composition comprising the peptide of claim as an active ingredient to a subject in need thereof.

12. The method of claim 11, wherein the composition further comprises an immune checkpoint inhibitor.

13. The method of claim 12, wherein the immune checkpoint inhibitor is one or more selected from the group consisting of a PD-1 inhibitor, a PD-L1 inhibitor, and a CTLA-4 inhibitor.

14. A method of treating cancer or inhibiting metastasis of cancer, comprising:
administering the composition comprising the peptide of claim 1 as an active ingredient to a subject in need thereof.

15. The method of claim 14, wherein the cancer is one or more selected from the group consisting of colorectal cancer, breast cancer, prostate cancer, melanoma, lung cancer, head and neck cancer, ovarian cancer, bladder cancer, stomach cancer, esophageal cancer, bile duct cancer, pancreatic cancer, liver cancer, cervical cancer, skin cancer, lymphoma, thyroid cancer, bone marrow cancer, endometrial cancer, and brain tumors.

16. The method of claim 14, wherein the peptide inhibits pre-metastatic niche formation.

17. A method of enhancing cancer immunotherapy sensitivity, comprising:
administering the composition comprising the peptide of claim 1 as an active ingredient into a subject in need thereof.

18. The method of claim 17, wherein the peptide improves the cancer immunotherapy activity of an immune checkpoint inhibitor.
